(19) United States
(12) Patent Application Publication
(10) Pub. No.: US 2017/0268008 A1
(43) Pub. Date: Sep. 21, 2017

(54) DIAGNOSTIC AND THERAPEUTIC TARGETING OF DNMT-1 ASSOCIATED RNA IN HUMAN CANCER

(71) Applicant: CASE WESTERN RESERVE UNIVERSITY, Cleveland, OH (US)

(72) Inventors: Ahmed Khalil, Cleveland, OH (US); Sanford Markowitz, Cleveland, OH (US)

(21) Appl. No.: 15/529,399

(22) PCT Filed: Nov. 24, 2015
(86) PCT No.: PCT/US15/62352

§ 371 Date: May 24, 2017

Related U.S. Application Data
(60) Provisional application No. 62/083,603, filed on Nov. 24, 2014.

(51) Int. Cl. C12N 15/113; C12Q 1/68

(52) U.S. Cl. CPC C12N 15/1137; C12Y 201/01037; C12Q 1/6886; C12N 2310/14; C12N 2310/141; C12N 2310/11; C12Q 2600/154; C12Q 2600/178

(57) ABSTRACT

A method for treating cancer in a subject in need thereof includes administering to cancer cells of the subject an agent effective to modulate the level of DNMT1-associated RNA and/or the interaction of DNMT1-associated RNA and DNMT1 in the cancer cells of the subject. Embodiments described herein relate to RNAs (e.g., IncRNAs) associated with DNA methyltransferase 1 (DNMTI-associated RNA) in human cancer cells, methods and compositions of modulating the levels of DNMTI-associated RNA and/or the interaction of DNMT1-associated RNA and DNMT1 in cancer cells of the subject to treat cancer cells or a subject in need thereof, and/or methods of measuring the expression profile of DNMT1 associated RNA to determine whether the subject has cancer or an increased risk of cancer and/or the efficacy of a therapeutic regimen agent.

### RELATED APPLICATION

This application claims priority from U.S. Provisional Application No. 62/083,603, filed Nov. 24, 2014, the subject matter of which is incorporated herein by reference in its entirety.

### BACKGROUND

Epigenetic regulation of gene expression in mammalian cells involve highly coordinated functions of chromatin remodeling complexes, histone modifying enzymes, DNA methyltransferases as well as chromatin readers. These interactions serve to activate or repress gene expression at specific genomic loci to ensure tissue specific gene expression patterns. However, how these ubiquitous epigenetic effectors are recruited, assembled and stabilized at specific genomic loci in distinct cell types is yet to be fully elucidated. We previously identified extensive interactions between human long intergenic non coding RNAs (lincRNAs) and several chromatin modifying complexes including the polycomb repressive complex (PRC2). These interactions are required for proper PRC2 mediated gene expression programs, and emerging evidence suggests a regulatory role of lincRNAs in recruiting and organizing PRC2 as well as other epigenetic complexes on chromatin.

The human genome encodes over 8,300 lincRNAs, which constitute a subclass of long non coding RNAs (lncRNAs) that are transcribed from genomic regions that do not overlap any protein coding genes. Although lincRNAs are capped, spliced and polyadenylated, many lincRNAs are retained in the nucleus. Recent genetic studies that knocked out several lincRNAs in mice provided in vivo evidence that some lincRNAs are required for embryonic development and tissue morphogenesis. Furthermore, recent studies have implicated lincRNAs in various human diseases including several cancers. In these studies, lincRNAs have been shown to exert either tumor suppressor or oncogenic effects sometimes by largely unknown mechanisms.

DNA methylation is an important epigenetic mark that is typically associated with repressed genes in mammalian cells. Three distinct DNA methyltransferases (DNMT1, DNMT3a and DNMT3b) are known to regulate DNA methylation patterns in mammals. Genome wide studies of DNA methylation in tumors have shown that the cancer genomes are largely hyopmethylated, however, the promoters of some tumor suppressors become hypermethylated. Currently, there is a great interest in understanding how DNA methylation patterns become deregulated in human cancers with the hope that these studies might lead to novel insights into tumorgenesis as well as future therapeutic interventions.

### SUMMARY

Embodiments described herein relate to RNAs (e.g., lncRNAs) associated with DNA methyltransferase 1 (DNMT1-associated RNA) in human cancer cells, methods and compositions of modulating the levels of DNMT1-associated RNA and/or the interaction of DNMT1-associated RNA and DNMT1 in cancer cells of the subject to treat cancer cells or a subject in need thereof, and/or methods of measuring the expression profile of DNMT1-associated RNA to determine whether the subject has cancer or an increased risk of cancer and/or the efficacy of a therapeutic regimen agent.

In some embodiments, cancer in a subject can be treated by administering an agent to cancer cells of the subject that is effective to modulate the level of DNMT1-associated RNA and/or the interaction of DNMT1-associated RNA and DNMT1 in the cancer cells. The cancer can be, for example, breast cancer or colon cancer. In other embodiments, the DNMT1-associated RNA can be DNMT1-associated long non-coding RNA.

In some embodiments, the agent administered to the cancer cells to treat cancer in the subject can be effective to decrease the level of DNMT1-associated RNA, which is over expressed in the cancer cells compared to normal cells. An agent effective to decrease the level of DNMT1-associated RNA, which is over expressed in the cancer cells, can include an RNA inhibitor of the DNMT1-associated RNA, such as siRNA, miRNA, stRNA, snRNA, shRNA, and antisense nucleic acids to the DNMT1-associated RNA.

In one example, the DNMT1-associated RNA that is over expressed or upregulated, can include at least one of linc-GATA5-1, linc-FAM84B-9, linc-OR10H4, linc-DUSP26-6, linc-CCDC40-1, linc-CSPP1, linc-ASPSCR1, linc-U2AF1-5, linc-BEAN1, linc-EFR3A-7, linc-SLC25A45-5, linc-SEMA3A, linc-CXXC4-1, linc-EFR3A-4, linc-JAKMIP3-3, linc-KIAA1755-4, linc-EPHB4, linc-GAD1-1, linc-IGFBP2-3, linc-CCDC122-4, linc-NADSYN1-2, linc-DUSP26-1, linc-EFR3A-5, linc-TCF20, linc-RSPH1-1, linc-DUXA-2, linc-RTEL1, linc-INO80, linc-UBE3C-2, linc-STIM2-1, linc-VEZF1, linc-GPR183-2, linc-WHAMM-1, linc-FRMPD1, linc-MIB2, linc-SERTAD2-4, linc-HAAO-4, linc-CDH5-3, linc-NDUFAF2-3, linc-PPM1J, linc-LY6H, linc-MKLN1-2, linc-SERPIND1, linc-TCP10-5, linc-PPIAL4F-1, linc-BIRC7-3, linc-S100B-2, linc-C1QTNF9B, linc-PXN, linc-SRL, linc-ZNF692-6, linc-BDH1-3, linc-RALGAPB, linc-MYOD1, linc-OR4F16-9, linc-MUC20-3, linc-BTBD6-1, linc-CDK13-1, linc-ZNF8-2, linc-HIST1H2AI-2, linc-OR7C2-1, linc-MZF1-2, linc-CMPK1-3, linc-ARHGAP28-9, linc-NACC1, linc-BMS1-4, linc-TCP11L2-1, linc-CANX-1, linc-KCTD7-2, linc-TMEM105-2, linc-MRPS31, linc-RGL4-1, linc-METTL14-1, linc-NDUFB4-5, linc-ARF5-2, linc-NBPF15-1, linc-PHF10, linc-NADSYN1-1, linc-TMEM183B-1, linc-CALCOCO2-3, linc-BDH1-2, linc-ADAMTSL4, linc-RPS7-1, linc-ATP6V1C2-4, linc-FSCN2-1, linc-TUBGCP3-2, linc-HOXD1, linc-TGFBRAP1, linc-NOP14-3, linc-IER5L-2, linc-ASPRV1-1, linc-TPT1-2, linc-OAF-6, linc-COX5B-3, linc-ZBED1-4, linc-HIST1H2AI-1, linc-CALCOCO2-2, linc-ARF6-1, linc-MAP7-BP, linc-LOC285033-4, linc-ZNF674, linc-HTR5A-1, linc-GPR179, linc-RPP40, linc-SATB2-2, linc-MUC20-2, linc-ZNF516-4, linc-STX17, linc-CDH6-7, linc-SERHL2-3, or linc-OR4F16-4.

In another example, the DNMT-1 associated RNA that is over expressed can include at least one of linc-TMEM169-3, linc-HEATR6-2, linc-TM4SF4-2, linc-DACT2-3, linc-SAFB-2, linc-PTPRS-2, linc-ZPBP2, linc-MGAT4A, linc-DUSP26-5, linc-CA5A-2, linc-MERTK-2, linc-ABCA5-3, linc-GUCA2B, linc-HOXD1, linc-FOXA1-2, linc-EGLN1-2, linc-LRRC49-4, linc-TMEM18-13, linc-ANKRD27, linc-LAMA1-5, linc-TMEM183B-1, linc-UGDH, linc-PKMYT1, linc-PPP1R1B, linc-WFDC2-2, linc-DYNC1LI1-2, linc-DHX37-22, linc-OAF-6, linc-ODF3B, linc-ARHGAP28-9, linc-DUSP26-6, linc-EVX2-8, linc-COPZ2, linc-DLGAP5-1, linc-XRCC4-3, linc-ZIC5, linc-KIN-5, linc-NACC1, linc-SERTAD2-4, linc-ASPSCR1, or linc-KIAA0232.

In yet another example, the DNMT-1 associated RNA can include at least one of linc-DUSP26-6, linc-ASPSCR1, linc-SERTAD2-4, linc-ARHGAP28-9, linc-NACC1, linc-TMEM183B-1, linc-HOXD1, or linc-OAF-6.

In other embodiments, the agent administered to the cancer cells to treat cancer in the subject can be effective to increase the level of DNMT1-associated RNA that is under expressed or downregulated in the cancer cells compared to normal cells. The agent can include, for example, a nucleic acid encoding the under expressed DNMT1-associated RNA that is administered to the cancer cells using, for example, an expression vector.

In one example, the DNMT1-associated RNA that is under expressed or down regulated in the cancer cells can include at least one of linc-SMAD3, linc-ANXA8L2-2, linc-TRAK1, linc-AP2B1-2, linc-UTRN, linc-TBX18, linc-GPR65-6, linc-STIL-2, linc-ENPP6-2, linc-GABRA5-6, linc-MRPS18C, linc-EGFL7-1, linc-KLHL31-2, linc-PSMA8-3, linc-ZNF404, linc-HMGB2, linc-OAF-4, linc-FRG1-5, linc-HIST1H3A, linc-TMEM56-3, linc-DBT-3, linc-GNAI1-2, linc-BCL2L10, linc-EPHA6-1, linc-PLDN, linc-GABRA5-5, linc-ACO1-2, linc-NEDD4L-1, linc-MTRNR2L1-2, linc-FAM155B, linc-GIMAP8-1, linc-MAGI2-3, linc-DHX37-17, linc-KLF6-3, linc-RAP1GAP2-1, linc-TMPRSS2-2, linc-C10orf57-3, linc-GPR157-3, linc-LAMA4-2, linc-STIM1, linc-RFC2-2, linc-MRGPRF-1, linc-DEFB105B-2, linc-CTDSP2-1, linc-PRPS1L1, linc-SLC19A1-4, linc-C10orf43-2, linc-COX4NB-8, linc-HES1-3, linc-FIGNL1, linc-OAF-2, linc-COX4NB-9, linc-FBXL5-2, linc-TMEM220-2, linc-KCNMB2-5, linc-KIAA0141, linc-DHX37-19, linc-RGMA-7, linc-ID2-1, linc-SHISA6-1, linc-SYT4-1, linc-TRIML2-5, linc-DHFRL1-4, linc-RGS9-1, linc-ODF2L, linc-SLC22A16, linc-ZPBP2, linc-AGMAT-3, linc-MT1B, linc-GRPEL1-1, linc-PFDN4-2, linc-OPRK1-4, linc-ZNF583-1, linc-PFDN4-3, linc-SAMSN1-3, linc-USP3-1, linc-SHISA6-2, linc-ADAM29-3, linc-ZEB2-7, linc-MLL5, linc-FOXF1-3, linc-BTBD3-3, linc-GPATCH2-9, linc-ARHGEF37-2, linc-KLF6-2, linc-CLMN-1, linc-FOXG1-4, linc-TAAR9-1, linc-GTPBP8, linc-ADAR, linc-SAFB-2, linc-CXorf49B-2, linc-SLCO2A1-1, linc-PTPRS-2, linc-EPCAM, linc-LPHN2-1, linc-AMN1, linc-FAM55D, linc-FAM75A6-4, or linc-PHOX2B-2.

In another example, the DNMT1-associated RNA that is under expressed or down regulated in the cancer cells can include at least one of linc-GHRH, linc-VPS36-1, linc-C20orf79, linc-AUTS2-5, linc-ISLR2-3, linc-ZNF692-6, linc-IER3IP1, linc-MANSC1, linc-CXADR-3, linc-ZFHX3-4, linc-ZNF404, linc-FAAH2-2, linc-CLRN2-1, linc-ATP6V1C2-3, linc-METTL14-2, linc-MAP1LC3B-2, linc-TCP11L2-1, linc-GARS-1, linc-NOP14-3, linc-ANKRD55-6, linc-ARFIP1-8, linc-C17orf87, linc-AMAC1, linc-SYT4-1, linc-HTR1D, linc-WNT7B-2, linc-MAP1LC3B2-2, linc-TP53TG3B-6, linc-TMEM105-2, linc-MICB, linc-PLGLB2, linc-OR4F16-9, linc-RBM10, linc-KIAA1712-5, linc-CBLB-6, linc-ATG2B-2, linc-ADI1, linc-SPRY3-1, linc-BEAN1, linc-SHOX-5, linc-WIPF3, linc-SHOX-4, linc-DCAF17-1, linc-TNFRSF14, linc-GPR65-6, linc-PHF10, linc-ZNF692-5, linc-POLR3A-1, linc-LOC389493-2, linc-AP2B1-2, linc-LRRTM3-3, linc-SFMBT1, linc-BTBD6-1, linc-MTHFD2, linc-PRSS42, linc-RGMB-1, linc-ITIH2-10, linc-TPBG-3, linc-TMEM194A, linc-FRG2C-3, linc-ZKSCAN1-1, linc-HEATR2-2, linc-CDK13-1, linc-GIMAP8-1, linc-FAM101A-2, linc-IFITM5, linc-LRRTM3-2, linc-METTL14-1, linc-GTPBP8, linc-KLF13-1, linc-SLC5A3-2, linc-BET3L, linc-TUBGCP3-2, linc-CDH1, linc-PTGR2, linc-CDK17-4, linc-COG3-3, linc-CBR1-1, linc-CCR8-3, linc-LOC150786, linc-FRG1-5, linc-GABRA5-7, linc-DYDC1-5, linc-CREB1-1, linc-LEPROTL1-7, linc-C6orf145-3, linc-HIST1H2AG-4, linc-THSD4, linc-HS3ST3A1-1, linc-KLRC1, linc-ZNF583-1, linc-ZNF253-2, linc-UTRN, linc-ATP6V1C2-4, linc-GRPEL1-1, linc-TTC7A-2, linc-COGS-1, linc-IFLTD1, linc-GALNTL5-1, linc-PCM1, linc-ISLR2-2, linc-DHCR7-2, linc-HES5-2, linc-USP8, linc-VPS8-2, linc-RGL4-1, linc-CEBPG, linc-CRH-2, linc-DR1, linc-UBQLN2, linc-MTRNR2L9-3, linc-ID2-3, linc-TMED5, linc-RAB23-4, linc-NDFIP2-1, linc-ARHGAP5, linc-WRNIP1-2, linc-ANKRD20A1-14, linc-TLN2-1, linc-VPS8-3, linc-CNTNAP4, linc-CHMP2B-1, linc-CXADR-2, linc-LOC285033-5, linc-ARHGAP28-2, linc-RGMA-7, linc-BMS1-3, linc-Clorf86, linc-CASP10-1, linc-SYNPO2-2, linc-FAM71F2-1, linc-TNKS-3, linc-MMADHC-3, linc-LEPROTL1-6, linc-BCL2L10, linc-OR5AU1, linc-SH3BGRL2-1, linc-TRIM43B-2, linc-ALG2-5, linc-C8orf79-2, linc-CEBPB-1, linc-RPS14, linc-RRP12, linc-FAM98A-3, linc-TACC3, linc-MPPE1-4, linc-IDH3B-1, linc-C8orf79-4, linc-DEFB105B-3, linc-HSF2-1, linc-EPT1, linc-RPGRIP1-3, linc-MUC20-1, linc-MAGI2-3, linc-SNTG2-3, linc-DEFB105B-2, linc-TCP10-4, linc-NAV3-1, linc-CSTB-6, linc-TCF7L2-3, linc-SPNS3, linc-FURIN, linc-HNRNPA1-3, linc-C1orf63, linc-SPAST-2, linc-EGFL7-1, linc-THBS2-3, linc-FRMPD1, linc-ITGB2-3, linc-TRIP11, linc-FER-1, linc-TMEM132D-1, linc-C14orf4-2, linc-GPATCH2-9, linc-ZNF236-4, linc-TTLL7-2, linc-APBA2-3, linc-ZNF32-5, linc-ALDH1A3-1, linc-GBP5-2, linc-DYNC1I1-2, linc-NR2F2-3, linc-B3GAT2-4, linc-CEP57-3, linc-CPPED1-3, linc-PHYHIPL, linc-C9orf170-1, linc-SPACA3, linc-FAM103A1, linc-INHBB, linc-ANKRD20A1-2, linc-CRP, linc-CPEB2-16, linc-PLCH2, linc-SHISA6-1, linc-ODZ3-5, linc-Clorf43-2, linc-HIST1H2AI-1, linc-BEND7-1, linc-CTSD-3, linc-RBKS-1, linc-PRSS38, linc-HAAO-6, linc-SLC5A9-4, linc-ZEB2-7, linc-FAM75A6-7, linc-DCT-2, linc-LY75, linc-DKK3-3, linc-TRPM5, linc-USPL1-1, linc-TPT1-2, linc-OBP2B, linc-C5orf38-4, linc-MMEL1-3, linc-GALNTL5-3, linc-ZDHHC6-2, linc-C5orf38-5, linc-HAAO-4, linc-CXorf36-3, linc-WDTC1, linc-LOC100129335-2, linc-DYNC2H1-4, linc-PEPD-1, linc-HDDC2-4, linc-TPT1-1, linc-USP16-5, linc-PICK1, linc-RHOXF1-3, linc-OXCT1-1, linc-BOD1-2, linc-ARHGEF37-2, linc-AQPEP, linc-PABPC4L-1, linc-MAP1LC3B-5, linc-TOX3-2, linc-FRMD6-2, linc-DEK-6, linc-ZFP64-5, linc-PRKAA2-8, linc-ABCA5-7, linc-PRKACG-2, and combinations thereof.

In yet another example, the DNMT1-associated RNA that is under expressed or down regulated in the cancer cells can include at least one of linc-AP2B1-2, linc-UTRN, linc-GPR65-6, linc-EGFL7-1, linc-ZNF404, linc-FRG1-5, linc-BCL2L10, linc-GIMAP8-1, linc-MAGI2-3, linc-DEFB105B-2, linc-C1orf43-2, linc-RGMA-7, linc-SHISA6-1, linc-SYT4-1, linc-GRPEL1-1, linc-ZNF583-1, linc-ZEB2-7, linc-GPATCH2-9, linc-ARHGEF37-2, or linc-GTPBP8.

Other embodiments described herein relate to a method of analyzing tissue in a subject having or suspected of having cancer. The method includes obtaining an expression profile from a sample of tissue obtained from the subject, wherein the expression profile comprises the level of at least one DNMT1-associated RNA selected from the group consisting of Tables 1, 2, 3, 4, 5, and 6. The expression profile from the sample is then compared to an expression profile of a control or standard. A decrease in the expression of the at least one DNMT1-associated RNA selected from Table 1, 3, or 5 and/or increase in the expression of the at least one DNMT1-associated RNA selected from Table 2, 4, or 6 is indicative of the subject having cancer or an increased risk of cancer. In some embodiments, the cancer is colon cancer or breast cancer.

Still other embodiments relate to a method of predicting whether a subject has cancer or an increased risk of cancer. The method includes obtaining an expression profile from a sample of tissue obtained from the subject, wherein the expression profile comprises the level of at least one DNMT1-associated RNA selected from the group consisting of Tables 1, 2, 3, 4, 5, and 6. The expression profile from the sample is then compared to an expression profile of a control or standard and whether the subject has cancer or an increased risk of cancer is predicted based on (i) deviation of the expression profile of the sample from a control or standard derived from a healthy individual or population of healthy individuals, or (ii) the similarity of expression profiles of the sample and a control or standard derived from an individual or population of individuals who have or have had the cancer. In some embodiments, a decrease in the expression of the at least one DNMT1-associated RNA selected from Table 1, 3, or 5 and/or an increase in the expression of the at least one DNMT1-associated RNA selected from Table 2, 4, or 6 is indicative of the subject having cancer or an increased risk of cancer. In some embodiments, the cancer is colon cancer or breast cancer.

Other embodiments relate to a method of monitoring a subject's response to a treatment regimen for cancer. The method includes administering a therapeutic regimen to the subject. An expression profile from a sample of cancer cells is obtained from the subject, wherein the expression profile comprises the level of at least one DNMT1-associated RNA selected from the group consisting of Tables 1, 2, 3, 4, 5, and 6. The expression profile from the sample is compared to an expression profile of a control or standard. An increase in the expression of the at least one DNMT1-associated RNA selected from Table 1, 3, or 5 and/or decrease in the expression of the at least one DNMT1-associated RNA selected from Table 2, 4, or 6 is indicative of an increased efficacy of the therapeutic regimen. In some embodiments, the cancer is colon cancer or breast cancer.

### BRIEF DESCRIPTION OF THE DRAWINGS

FIGS. 1(A-H) illustrate: (A) Outline of the experimental strategy utilized to identify DNMT1-associated RNAs. (B) Western blot analysis using an anti-flag-DNMT1 antibody confirms the specific immunoprecipitation (IP) of DNMT1, but not other highly abundant nuclear proteins (histone H3, U1-70K). An IP with anti-IgG antibody demonstrates that there is no detectable background. (C) Heatmap of lncRNAs in input sample versus each of the three biological replicates of DNMT1 RIPs. (D) Heatmap of mRNAs in input versus the three biological replicates of DNMT1 RIPs. (E) Pie chart showing the number of DNMT1-associated lncRNAs versus all lncRNAs expressed in input. (F) Pie chart showing the number of DNMT1-associated mRNAs versus mRNAs expressed in input. (G-H) Graphs show the expression levels of DNMT1-bound lncRNAs and mRNAs versus non-bound lncRNAs and mRNAs in HCT116 cells.

FIGS. 2(A-D) illustrate: (A) A graph showing quantitative real-time PCR (qRT-PCR) of DACOR1 across a panel of human normal tissues. (B) RNA in situ hybridization of the expression of DACOR1 in human colon tissues and colon crypts as one of the major cell types that express it. Close examination of colon cells (small panel) reveals that DACOR1 is retained in the nucleus and potentially interacts with chromatin. (C) Expression analysis of DACOR1 in a cohort of 22 colon cancer tumors versus 22 matched normal tissues in RNA-seq datasets obtained from TCGA demonstrates that DACOR1 is down-regulated in colon tumors. (D) Examining the expression of DACOR1 by qRT-PCR in 8 normal colon samples and 21 patient-derived colon cancer cell lines with limited passage in culture demonstrates that DACOR1 is highly repressed in most colon cancer cells.

FIGS. 3(A-E) illustrate: (A) A graph showing validation of the interaction between DNMT1 and DACOR1 by RIP-qPCR. DACOR1 shows a 7-fold enrichment in flag-DNMT1 RIP over IgG RIP. (B) The highly abundant nuclear RNA U1 shows no enrichment in flag-DNMT1 RIP versus IgG RIP. (C) A schematic drawing showing, induction of DACOR1 expression in two distinct patient-derived colon cancer cell lines (V481 and V852) enhances DNA methylation at multiple genomic loci in trans. (D) Induction of DACOR1 expression in patient-derived colon cancer cell lines (V866 and V852) results in up-regulation of tight junction protein 1 (TJP1), suggesting a potential role for DACOR1 in maintaining an epithelial state of colon cells. (E) The colon cancer cell lines V481, V852 and V866 were transduced with either a control or DACOR1 lentivirus.

FIGS. 4 (A-D) illustrate: (A) qRT-PCR confirmations of RNA-seq data that DACOR1 represses several genes (SMAD6, FST and INHBE) involved in the repression of the TGF-beta/BMP signaling pathway. (B) qRT-PCR validations of RNA-seq data that DACOR1 represses the expression of key genes that are involved in amino acid biosynthesis and metabolism; (C) Western blot analyses demonstrate that DACOR1 induction leads to the repression of PHGDH and CBS but does not affect PKM2 or Actin protein levels in V852 cells; (D) Induction of DACOR1 reduces the activity of PKM2, which is known to be dependent on serine, without affecting overall PKM2 protein levels.

FIGS. 5(A-C) illustrate: (A) Confirmation of DACOR1 pull down from crosslinked cell lysates by specific complementary probes, in comparison with non-specific probes. (B) Intersection of DACOR1 genome occupancy sites near annotated protein-coding genes identified in this study by ChIRP-seq and differentially methylated regions (DMRs) in colon tumors/normal colon identified by Simmer et al. reveals a significant overlap. This further supports the role of DACOR1, via its interaction with DNMT1, in regulating genome-wide DNA methylation. (C) A proposed model of how DNMT1-DACOR1 interactions regulate DNA methylation and gene expression.

FIG. 6 illustrates expression analysis of DACOR1 by qRT-PCR in normal colon vs patient-derived colon cancer cell lines represented as a cluster graph.

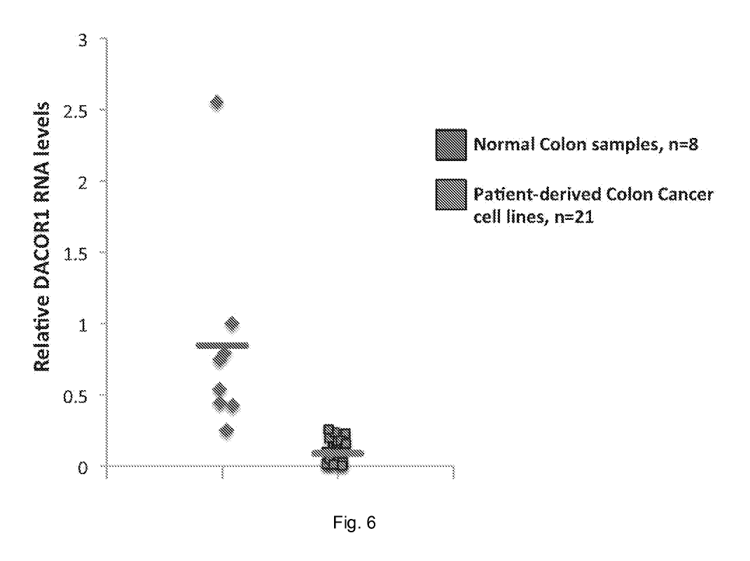

FIGS. 7(A-B) illustrates (A) Expression analysis by qRT-PCR of DACOR1 in normal colon, two colon cancer cell lines transduced with a control lentivirus, and same two cell lines transduced with a DACOR1 lentivirus. (B) RNA in situ demonstrates lack of DACOR1 expression in colon cancer cells (left panel), and the appropriate induction and nuclear localization of DACOR1 using a lentivirus (right panel).

FIG. 8 illustrates DACOR1 induction results in decreased growth of colon cancer cells. A field view of colon cancer cells that were transduced with either a control or DACOR1 lentivirus. We quantified the effect of DACOR1 on the growth of colon cancer cells using colony formation assays.

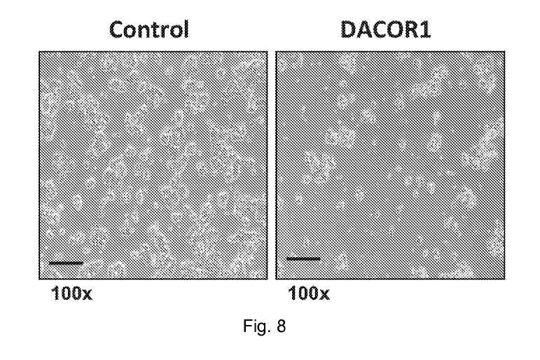

FIGS. 9(A-B) illustrate: (A-B) qRT-PCR expression analysis of DACOR1 in the colon cancer cell lines V703 and V425 post transduction with either a control or DACOR1 lentivirus (CMV promoter). (C-D) DACOR1 has minor effects on colony formation in V703 and V425 cells. These are colon cancer cell lines that maintain some endogenous levels of DACOR1 expression.

FIGS. 10(A-C) illustrate graphs showing linc-SAFB-2 (TINCR) is upregulated in breast cancer (A-B) and knock down of TINCR results in reduced tumor growth (C).

### DETAILED DESCRIPTION

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention which will be limited only by the appended claims.

The term “pharmaceutically acceptable carrier” refers to any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “subject” refers to any organism or animal to whom treatment or prophylaxis treatment is desired. Such animals include mammals, preferably a human. The term “subject” also refers to any living organism from which a biological sample can be obtained. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” is also intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species. In another embodiment, the subject is an experimental animal or animal substitute as a disease model.

The term “mammal” or “mammalian” are used interchangeably herein, and encompass their normal meaning. While the methods and compositions described herein are most desirably intended for efficacy in humans, they may also be employed in domestic mammals such as canines, felines, and equines, as well as in mammals of particular interest, e.g., zoo animals, farmstock, transgenic animals, rodents and the like.

The terms “gene silencing” or “gene silenced” in reference to an activity of a RNAi molecule, for example a siRNA or miRNA refers to a decrease in the mRNA level in a cell for a heterologous target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one embodiment, the mRNA levels are decreased by at least about 70%, about 80%, about 90%, about 95%, about 99%, about 100%. As used herein, the “reduced” or “gene silencing” refers to lower, preferably significantly lower, more preferably the expression of the nucleotide sequence is not detectable.

The term “double-stranded RNA” molecule, “RNAi molecule”, or “dsRNA” molecule refers to a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule. In some embodiments, the terms refer to a double-stranded RNA molecule capable, when expressed, is at least partially reducing the level of the mRNA of the heterologous target gene. In particular, the RNAi molecule is complementary to a synthetic RNAi target sequence located in a non-coding region of the heterologous target gene.

The terms “RNA interference”, “RNAi”, and “dsRNAi” are used interchangeably herein and refer to nucleic acid molecules capable of gene silencing.

The term “RNAi” refers to any type of interfering RNA, including siRNAi, shRNAi, stRNAi, endogenous microRNA and artificial microRNA. For instance, it includes sequences previously identified as siRNA, regardless of the mechanism of down-stream processing of the RNA. The term “siRNA” also refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 10-50 nucleotides in length (e.g., each complementary sequence of the double stranded siRNA is about 10-22 nucleotides in length, and the double stranded siRNA is about 10-22 base pairs in length, preferably about 19-22 base nucleotides, preferably about 17-19 nucleotides in length, e.g., 10, 11, 12, 13, 14, 15, 16, 17, 18, 19, 20, 21, or 22 nucleotides in length).

The terms “shRNA” or “small hairpin RNA” (also called stem loop) is a type of siRNA. In one embodiment, these shRNAs are composed of a short, e.g., about 10 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The term a “stem-loop structure” refers to a nucleic acid having a secondary structure that includes a region of nucleotides, which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e., not include any mismatches. In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof. The actual primary sequence of nucleotides within the stem-loop structure is not critical as long as the secondary structure is present. As is known in the art, the secondary structure does not require exact base-pairing. Thus, the stem may include one or more base mismatches. Alternatively, the base pairing may not include any mismatches.

The term “hairpin RNA” refers to any self-annealing double stranded RNA molecule. In its simplest representation, a hairpin RNA consists of a double stranded stem made up by the annealing RNA strands, connected by a single stranded RNA loop, and is also referred to as a “pan-handle RNA”. However, the term “hairpin RNA” is also intended to encompass more complicated secondary RNA structures comprising self-annealing double stranded RNA sequences, but also internal bulges and loops. The specific secondary structure adapted will be determined by the free energy of the RNA molecule, and can be predicted for different situations using appropriate software such as FOLDRNA (Zuker and Stiegler (1981) Nucleic Acids Res 9(1):133-48; Zuker, M. (1989) Methods Enzymol. 180, 262-288).

The term “agent” refers to any entity which is normally absent or not present at the levels being administered, in the cell. An agent may be selected from a group comprising; chemicals; small molecules; nucleic acid sequences; nucleic acid analogues; proteins; peptides; aptamers; antibodies; or fragments thereof. A nucleic acid sequence may be RNA or DNA, and may be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. The agent may be applied to the media, where it contacts the cell and induces its effects. Alternatively, the agent may be intracellular within the cell as a result of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The terms “a reduction” of the level of an RNA, mRNA, rRNA, tRNA, or lncRNA includes a decrease in the level of the RNA, mRNA, rRNA, tRNA, or lncRNA in the cell or organism. “At least a partial reduction” of the level of the RNA, mRNA, rRNA, tRNA or lncRNA means that the level is reduced at least about 10%, at least about 25%, at least 50% or more relative to a cell or organism in which the level of RNA, mRNA, rRNA, tRNA or lncRNA is not reduced by some means. “A substantial reduction” of the level of RNA, mRNA, rRNA, tRNA or lncRNA means that the level is reduced at least about 75%, at least about 85% or more. The reduction can be determined by methods with which the skilled worker is familiar Thus, the reduction can be determined for example by reverse transcription (quantitative RT-PCR), ELISA (enzyme-linked immunosorbent assay), Western blotting, radioimmunoassay (RIA) or other immunoassays and fluorescence-activated cell analysis (FACS).

In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of at least 10 nucleotides, or at least 11, 12, 13, 14, 15, 16, 17, 18, 19, 20, 21, 22 or up to 50 nucleotides of the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J MoI. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

The term “substantially identical”, when used herein with respect to a nucleotide sequence, means a nucleotide sequence corresponding to a reference or target nucleotide sequence, wherein the percentage of identity between the substantially identical nucleotide sequence and the reference or target nucleotide sequence is at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of 10-22 nucleotides, such as at least 10, 11, 12, 13, 14, 15, 16, 17, 18, 19, 20, 21, 22 or up to 50 nucleotides of a nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J MoI. Biol. 48: 443-453; as defined above). A nucleotide sequence “substantially identical” to a reference nucleotide sequence hybridizes to the exact complementary sequence of the reference nucleotide sequence (i.e., its corresponding strand in a double-stranded molecule) under low stringency conditions, preferably medium stringency conditions, most preferably high stringency conditions (as defined above). Homologues of a specific nucleotide sequence include nucleotide sequences that encode an amino acid sequence that is at least 24% identical, at least 35% identical, at least 50% identical, at least 65% identical to the reference amino acid sequence, as measured using the parameters described above, wherein the amino acid sequence encoded by the homolog has the same biological activity as the protein encoded by the specific nucleotide. The term “substantially non-identical” refers to a nucleotide sequence that does not hybridize to the nucleic acid sequence under stringent conditions.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, inderdisposion, affection.

The terms “malignancy” or “cancer” are used interchangeably herein and refers to any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer. The term “tumor” or “tumor cell” are used interchangeably herein, refers to the tissue mass or tissue type of cell that is undergoing abnormal proliferation.

The term “biological sample” or “sample” as used herein refers to a cell or population of cells or a quantity of tissue or fluid from a subject. Most often, the sample has been removed from a subject, but the term “biological sample” can also refer to cells or tissue analyzed in vivo, i.e., without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g., buccal scrapes), whole blood, plasma, serum, urine, saliva, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g., isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term “tissue” is intended to include intact cells, blood, blood preparations such as plasma and serum, bones, joints, muscles, smooth muscles, and organs.

The term “treatment” refers to any treatment of a pathologic condition in a subject, particularly a human subject, and includes one or more of the following: (a) preventing a pathological condition from occurring in a subject which may be predisposition to the condition but has not yet been diagnosed with the condition and, accordingly, the treatment constitutes prophylactic treatment for the disease or condition; (b) inhibiting the pathological condition, i.e., arresting its development, (c) relieving the pathological condition, i.e. causing a regression of the pathological condition; or (d) relieving the conditions mediated by the pathological condition.

The term “computer” refers to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” is used interchangeably herein with “program” and refers to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “optional” or “optionally” means that the subsequent described event, circumstance or substituent may or may not occur, and that the description includes instances where the event or circumstance occurs and instances where it does not.

Other than in the operating examples, or where otherwise indicated, all numbers expressing quantities of ingredients or reaction conditions used herein should be understood as modified in all instances by the term “about.” The term “about” when used in connection with percentages can mean.+−0.1%.

As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof.

In this specification and the appended claims, the singular forms “a,” “an,” and “the” include plural references unless the context clearly dictates otherwise, and therefore “a” and “an” are used herein to refer to one or to more than one (i.e., at least one) of the grammatical object of the article. By way of example, “an element” means one element or more than one element, and reference to a composition for delivering “an agent” includes reference to one or more agents.

Compositions or methods “comprising” one or more recited elements may include other elements not specifically recited. For example, a composition that comprises an inhibitor of HOTAIR encompasses both an inhibitor of HOTAIR but may also include other agents or other components. By way of further example, a composition that comprises elements A and B also encompasses a composition consisting of A, B and C. The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.”

Embodiments described herein relate to RNAs (e.g., lncRNAs, lincRNAs, and mRNAs) associated with DNA methyltransferase DNMT1 (DNMT1-associated RNA) in human cancer cells, methods and compositions of modulating the levels of DNMT1-associated RNA and/or the interaction of DNMT1-associated RNA with DNMT1 in the cancer cells of the subject to treat cancer cells or a subject in need thereof, and/or methods of measuring the expression profile of DNMT1-associated RNA to determine whether the subject has cancer or an increased risk of cancer and/or measure the efficacy of a therapeutic regimen or agent. It was discovered that human DNMT1 can interact or associate with non-coding RNAs, such as human lncRNAs, suggesting that in addition to histone modifications, DNA methylation is also indirectly regulated by non-coding RNA, such as lncRNAs. DNA methylation is an important epigenetic mark for the regulation of gene expression in mammalian cells from early embryonic development to fully differentiated post-mitotic cells. The fact that DNMT1 associates with certain lncRNAs suggests that these lncRNAs can influence DNMT1 genomic occupancy and/or activities, thereby indirectly regulating the methylome. Thus, deregulation of one or more of DNMT1-associated lncRNAs in human disease, such as cancer, would lead to changes in DNA methylation patterns and significant changes in gene expression without any detectable changes in DNMT1 expression levels.

It was found that under expressed or over-expressed DNMT1-associated RNA in cancer cells compared to normal cells can be targeted by agents that promote induction or inhibition, respectively, of the under expressed or over-expressed DNMT1-associated RNA to change DNA methylation patterns within the cancer cells without affecting DNMT1 protein levels in the cancer cells. This change in methylation pattern caused by such induction or inhibition can be used to inhibit cancer cell growth, proliferation, migration and/or metastasis.

Without wishing to be bound by theory, it is believe that DNMT1-associated RNA mediated changes in DNA methylation patterns are potentially caused by DNMT1-associated RNA recruitment of DNMT1 to specific sites of the genome, similar to what has been observed of lncRNA-mediated recruitment of histone-modifying enzymes. Also, DNMT1-associated RNA can potentially affect DNMT1 activity at specific CpG sites, by regulating protein components of the DNMT1 macromolecular protein complex.

As shown in the Examples, gene expression analyses of DNMT1-associated RNA demonstrated that colon cancer cell lines and breast cancer cell lines dramatically repress expression of some DNMT1-associated RNA but dramatically promote expression of others compared to normal colon cells and breast cells. Restoring DNMT1-associated RNA expression levels to those similar to normal cells resulted in reduced growth of the cancer cells, potentially via the modulation of several pathways.

By way of example, the DNMT1-associated RNA, DACOR1 interacts with specific genomic loci and potentially recruits DNMT1 to establish DNA methylation patterns and/or regulate gene expression. The DNMT1-DACOR1 axis results in modulating the expression of many genes, directly and indirectly, including those involved in amino acid metabolism. DACOR1 down-regulates the expression of several genes that inhibit TGF-β/BMP signaling and thus potentially enhances TGF-β/BMP signaling, which is known to exert a tumor-suppressor activity in the colon. DACOR1 also down-regulates several genes involved in metabolism including de novo serine biosynthesis (e.g., PHGDH, PSAT1). Serine is an essential precursor for the synthesis of proteins, nucleic acid and lipids; thus, it is critical for cancer cell growth. Furthermore, DACOR1 induction is sufficient to attenuate pyruvate kinase M2 (PKM2) activity, which is highly dependent on serine. PKM2 has been recently implicated as a key gene in cancer metabolism; thus, the identification of a lncRNA that attenuates its activity, although indirectly, can provide a therapeutic window in cancer biology. Lastly, DACOR1-mediated down-regulation of CBS, the deficiency of which is known to lead to increased levels of methionine and, consequently, SAM, the key methyl donor utilized by DNA methyl transferases to methylate DNA, is also highly significant. These findings suggest that DNMT1, via its interaction with RNA, such as DACOR1, indirectly regulates the cellular levels of SAM and, subsequently, genome-wide DNA methylation and that reestablishing down regulated DACOR-1 levels in cancer cells to levels found in comparable normal cells can be used to inhibit cancer growth and proliferation.

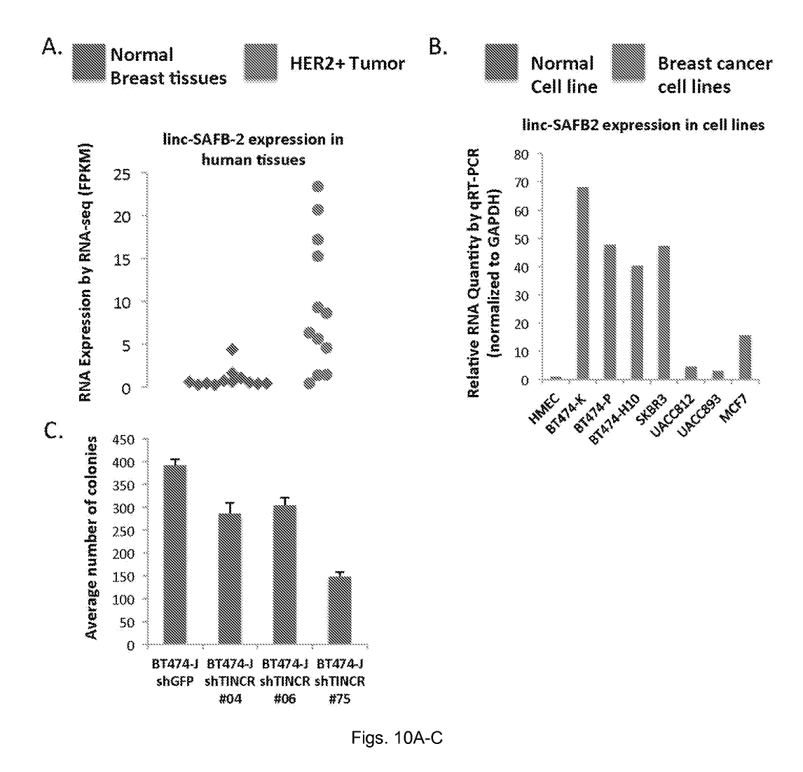

Moreover, it was found that linc-SAFB-2 (TINCR) is upregulated in HER2-positive breast cancer as compared to matched normal tissues. Knock down of TINCR results in HER2 positive breast cancer results in reduced breast cancer and proliferation (FIG. 10) growth.

Accordingly, DNMT1-associated RNA can regulate the human methylome and the genome-wide changes in DNA methylation across numerous cancer types. Modulating aberrant levels of DNMT1-associated RNA and/or the interaction of DNMT1-associated RNA and DNMT1 in the cancer cells of the subject can be used to treat cancer cells or a subject in need thereof. Furthermore, as many DNMT1-associated RNAs have tissue-specific expression patterns, they can serve as biomarkers for analyzing, diagnosing, prognosing, or determining the prognosis of cancer cells, as well as for determining or monitoring therapeutic strategies or regimens for cancer cell treatment in a subject with potentially less side-effects.

Tables 1-6 list DNMT1-associated RNAs include DACOR1 (linc-SMAD3) whose expression levels are downregulated or upregulated, respectively, in colon and breast cancer cells compared to normal colon and breast cells. As discussed in the Example, a RIP protocol was used to identify potential interactions between DNMT1 and RNAs. Isolated co-immunoprecipitated RNAs were quantified and RNA-seq libraries from independent biological replicates of DNMT1 RIPs were sequenced and mapped to the human genome. Fpkm values for mRNAs and lncRNAs were detected in the input sample and each of the biological replicates of DNMT1 RIP-seq. The average fpkm of each transcript in the biological replicates of DNMT1 RIP-seq was divided by the fpkm in the input sample to generate fold changes. LncRNAs and mRNAs were identified as DNMT1-associated RNAs based on a 2-fold change or higher. To rule out non-specific co-immunoprecipitation of highly abundant RNAs with DNMT1, the expression of all DNMT1-bound were compared versus DNMT1-unbound lncRNAs and mRNAs to determine downregulated or upregulated lincRNAs in the cancer cell compared to normal cells. The downregulated and upregulated DNMT1-associated RNA so identified can be used in methods and compositions of treating a subject with cancer in need thereof, and/or methods of measuring the expression profile of DNMT1-associated RNA to determine whether the subject has cancer or an increased risk of cancer and/or measure the efficacy of a therapeutic regimen or agent.

TABLE 1
DOWNREGULATED IN COLON TUMORS

| lncRNA | [T]/[N] | SEQ ID NO. |
|---|---|---|
| linc-ANXA8L2-2 | 0.499 | SEQ ID NO: 1 |
| linc-TRAK1 | 0.498 | SEQ ID NO: 2 |
| line-AP2B1-2 | 0.498 | SEQ ID NO: 3 |
| linc-UTRN | 0.498 | SEQ ID NO: 4 |
| linc-TBX18 | 0.497 | SEQ ID NO: 5 |
| linc-GPR65-6 | 0.490 | SEQ ID NO: 6 |
| linc-STIL-2 | 0.488 | SEQ ID NO: 7 |
| linc-ENPP6-2 | 0.487 | SEQ ID NO: 8 |
| linc-GABRA5-6 | 0.486 | SEQ ID NO: 9 |
| linc-MRPS18C | 0.483 | SEQ ID NO: 10 |
| linc-EGFL7-1 | 0.482 | SEQ ID NO: 11 |
| linc-KLHL31-2 | 0.479 | SEQ ID NO: 12 |
| linc-PSMA8-3 | 0.479 | SEQ ID NO: 13 |
| linc-ZNF404 | 0.472 | SEQ ID NO: 14 |
| linc-HMGB2 | 0.470 | SEQ ID NO: 15 |
| linc-OAF-4 | 0.470 | SEQ ID NO: 16 |
| linc-FRG1-5 | 0.468 | SEQ ID NO: 17 |
| linc-HIST1H3A | 0.456 | SEQ ID NO: 18 |
| linc-TMEM56-3 | 0.449 | SEQ ID NO: 19 |
| linc-DBT-3 | 0.440 | SEQ ID NO: 20 |
| linc-GNAI1-2 | 0.437 | SEQ ID NO: 21 |
| linc-BCL2L10 | 0.435 | SEQ ID NO: 22 |
| linc-EPHA6-1 | 0.434 | SEQ ID NO: 23 |
| linc-PLDN | 0.431 | SEQ ID NO: 24 |
| linc-GABRA5-5 | 0.430 | SEQ ID NO: 25 |
| linc-ACO1-2 | 0.430 | SEQ ID NO: 26 |
| linc-NEDD4L-1 | 0.425 | SEQ ID NO: 27 |
| linc-MTRNR2L1-2 | 0.417 | SEQ ID NO: 28 |
| linc-FAM155B | 0.412 | SEQ ID NO: 29 |
| linc-GIMAP8-1 | 0.410 | SEQ ID NO: 30 |
| linc-MAGI2-3 | 0.409 | SEQ ID NO: 31 |
| linc-DHX37-17 | 0.408 | SEQ ID NO: 32 |
| linc-KLF6-3 | 0.403 | SEQ ID NO: 33 |
| linc-RAP1GAP2-1 | 0.400 | SEQ ID NO: 34 |
| linc-TMPRSS2-2 | 0.400 | SEQ ID NO: 35 |
| linc-C10orf57-3 | 0.397 | SEQ ID NO: 36 |
| linc-GPR157-3 | 0.395 | SEQ ID NO: 37 |
| linc-LAMA4-2 | 0.391 | SEQ ID NO: 38 |
| linc-STIM1 | 0.390 | SEQ ID NO: 39 |
| linc-RFC2-2 | 0.387 | SEQ ID NO: 40 |
| linc-MRGPRF-1 | 0.385 | SEQ ID NO: 41 |
| linc-DEFB105B-2 | 0.384 | SEQ ID NO: 42 |
| linc-CTDSP2-1 | 0.382 | SEQ ID NO: 43 |
| linc-PRPS1L1 | 0.381 | SEQ ID NO: 44 |
| linc-SLC19A1-4 | 0.380 | SEQ ID NO: 45 |
| linc-C1orf43-2 | 0.379 | SEQ ID NO: 46 |
| linc-COX4NB-8 | 0.377 | SEQ ID NO: 47 |
| linc-HES1-3 | 0.374 | SEQ ID NO: 48 |
| linc-FIGNL1 | 0.374 | SEQ ID NO: 49 |
| linc-OAF-2 | 0.362 | SEQ ID NO: 50 |
| linc-COX4NB-9 | 0.360 | SEQ ID NO: 51 |
| linc-FBXL5-2 | 0.350 | SEQ ID NO: 52 |
| linc-TMEM220-2 | 0.348 | SEQ ID NO: 53 |
| linc-KCNMB2-5 | 0.339 | SEQ ID NO: 54 |
| linc-KIAA0141 | 0.338 | SEQ ID NO: 55 |
| linc-DHX37-19 | 0.329 | SEQ ID NO: 56 |
| linc-RGMA-7 | 0.327 | SEQ ID NO: 57 |
| linc-ID2-1 | 0.324 | SEQ ID NO: 58 |
| linc-SHISA6-1 | 0.322 | SEQ ID NO: 59 |
| linc-SYT4-1 | 0.319 | SEQ ID NO: 60 |
| linc-TRIML2-5 | 0.314 | SEQ ID NO: 61 |
| linc-DHFRL1-4 | 0.303 | SEQ ID NO: 62 |
| linc-RGS9-1 | 0.298 | SEQ ID NO: 63 |
| linc-ODF2L | 0.296 | SEQ ID NO: 64 |
| linc-SLC22A16 | 0.294 | SEQ ID NO: 65 |
| linc-ZPBP2 | 0.272 | SEQ ID NO: 66 |
| linc-AGMAT-3 | 0.264 | SEQ ID NO: 67 |
| linc-MT1B | 0.252 | SEQ ID NO: 68 |
| linc-GRPEL1-1 | 0.238 | SEQ ID NO: 69 |
| linc-PFDN4-2 | 0.236 | SEQ ID NO: 70 |
| linc-OPRK1-4 | 0.232 | SEQ ID NO: 71 |
| linc-ZNF583-1 | 0.231 | SEQ ID NO: 72 |
| linc-PFDN4-3 | 0.231 | SEQ ID NO: 73 |
| linc-SAMSN1-3 | 0.230 | SEQ ID NO: 74 |
| linc-USP3-1 | 0.214 | SEQ ID NO: 75 |
| linc-SHISA6-2 | 0.213 | SEQ ID NO: 76 |
| linc-ADAM29-3 | 0.210 | SEQ ID NO: 77 |
| linc-ZEB2-7 | 0.209 | SEQ ID NO: 78 |
| linc-MLL5 | 0.191 | SEQ ID NO: 79 |
| linc-FOXF1-3 | 0.191 | SEQ ID NO: 80 |
| linc-BTBD3-3 | 0.188 | SEQ ID NO: 81 |
| linc-GPATCH2-9 | 0.187 | SEQ ID NO: 82 |
| linc-ARHGEF37-2 | 0.180 | SEQ ID NO: 83 |
| linc-KLF6-2 | 0.176 | SEQ ID NO: 84 |
| linc-CLMN-1 | 0.169 | SEQ ID NO: 85 |
| linc-FOXG1-4 | 0.161 | SEQ ID NO: 86 |
| linc-TAAR9-1 | 0.160 | SEQ ID NO: 87 |
| linc-GTPBP8 | 0.149 | SEQ ID NO: 88 |
| linc-ADAR | 0.127 | SEQ ID NO: 89 |
| linc-SAFB-2 | 0.120 | SEQ ID NO: 90 |
| linc-CXorf49B-2 | 0.108 | SEQ ID NO: 91 |
| linc-SLCO2A1-1 | 0.098 | SEQ ID NO: 92 |
| linc-PTPRS-2 | 0.095 | SEQ ID NO: 93 |
| linc-EPCAM | 0.092 | SEQ ID NO: 94 |
| linc-LPHN2-1 | 0.085 | SEQ ID NO: 95 |
| linc-AMN1 | 0.085 | SEQ ID NO: 96 |
| linc-FAM55D | 0.065 | SEQ ID NO: 97 |
| linc-FAM75A6-4 | 0.057 | SEQ ID NO: 98 |
| linc-PHOX2B-2 | 0.018 | SEQ ID NO: 99 |
| linc-SMAD3 | | SEQ ID NO: 522 |

TABLE 2
UPREGULATED IN COLON TUMORS

| lncRNA | [T]/[N] | SEQ ID NO. |
|---|---|---|
| linc-GATA5-1 | 163.265 | SEQ ID NO: 100 |
| linc-FAM84B-9 | 34.817 | SEQ ID NO: 101 |
| linc-OR10H4 | 17.546 | SEQ ID NO: 102 |
| linc-DUSP26-6 | 15.893 | SEQ ID NO: 103 |
| linc-CCDC40-1 | 11.889 | SEQ ID NO: 104 |
| linc-CSPP1 | 11.092 | SEQ ID NO: 105 |
| linc-ASPSCR1 | 7.932 | SEQ ID NO: 106 |
| linc-U2AF1-5 | 7.298 | SEQ ID NO: 107 |
| linc-BEAN1 | 6.947 | SEQ ID NO: 108 |
| linc-EFR3A-7 | 6.846 | SEQ ID NO: 109 |
| linc-SLC25A45-5 | 6.544 | SEQ ID NO: 110 |
| linc-SEMA3A | 6.024 | SEQ ID NO: 111 |
| linc-CXXC4-1 | 5.815 | SEQ ID NO: 112 |
| linc-EFR3A-4 | 5.538 | SEQ ID NO: 113 |
| linc-JAKMIP3-3 | 5.406 | SEQ ID NO: 114 |
| linc-KIAA1755-4 | 5.392 | SEQ ID NO: 115 |
| linc-EPHB4 | 5.247 | SEQ ID NO: 116 |
| linc-GAD1-1 | 5.172 | SEQ ID NO: 117 |
| linc-IGFBP2-3 | 5.068 | SEQ ID NO: 118 |
| linc-CCDC122-4 | 4.934 | SEQ ID NO: 119 |
| linc-NADSYN1-2 | 4.788 | SEQ ID NO: 120 |
| linc-DUSP26-1 | 4.725 | SEQ ID NO: 121 |
| linc-EFR3A-5 | 4.613 | SEQ ID NO: 122 |
| linc-TCF20 | 4.446 | SEQ ID NO: 123 |
| linc-RSPH1-1 | 4.343 | SEQ ID NO: 124 |
| linc-DUXA-2 | 4.325 | SEQ ID NO: 125 |
| linc-RTEL1 | 4.313 | SEQ ID NO: 126 |
| linc-INO80 | 4.085 | SEQ ID NO: 127 |
| linc-UBE3C-2 | 4.032 | SEQ ID NO: 128 |
| linc-STIM2-1 | 3.860 | SEQ ID NO: 129 |
| linc-VEZF1 | 3.857 | SEQ ID NO: 130 |
| linc-GPR183-2 | 3.829 | SEQ ID NO: 131 |
| linc-WHAMM-1 | 3.755 | SEQ ID NO: 132 |
| linc-FRMPD1 | 3.724 | SEQ ID NO: 133 |
| linc-MIB2 | 3.472 | SEQ ID NO: 134 |
| linc-SERTAD2-4 | 3.464 | SEQ ID NO: 135 |
| linc-HAAO-4 | 3.434 | SEQ ID NO: 136 |
| linc-CDH5-3 | 3.306 | SEQ ID NO: 137 |
| linc-NDUFAF2-3 | 3.298 | SEQ ID NO: 138 |
| linc-PPM1J | 3.164 | SEQ ID NO: 139 |
| linc-LY6H | 3.159 | SEQ ID NO: 140 |
| linc-MKLN1-2 | 3.151 | SEQ ID NO: 141 |
| linc-SERPIND1 | 3.136 | SEQ ID NO: 142 |
| linc-TCP10-5 | 3.132 | SEQ ID NO: 143 |
| linc-PPIAL4F-1 | 3.022 | SEQ ID NO: 144 |
| linc-BIRC7-3 | 3.022 | SEQ ID NO: 145 |
| linc-S100B-2 | 2.974 | SEQ ID NO: 146 |
| linc-C1QTNF9B | 2.932 | SEQ ID NO: 147 |
| linc-PXN | 2.931 | SEQ ID NO: 148 |
| linc-SRL | 2.931 | SEQ ID NO: 149 |
| linc-ZNF692-6 | 2.927 | SEQ ID NO: 150 |
| linc-BDH1-3 | 2.918 | SEQ ID NO: 151 |
| linc-RALGAPB | 2.908 | SEQ ID NO: 152 |
| linc-MYOD1 | 2.890 | SEQ ID NO: 153 |
| linc-OR4F16-9 | 2.861 | SEQ ID NO: 154 |
| linc-MUC20-3 | 2.854 | SEQ ID NO: 155 |
| linc-BTBD6-1 | 2.835 | SEQ ID NO: 156 |
| linc-CDK13-1 | 2.821 | SEQ ID NO: 157 |
| linc-ZNF8-2 | 2.789 | SEQ ID NO: 158 |
| linc-HIST1H2AI-2 | 2.748 | SEQ ID NO: 159 |
| linc-OR7C2-1 | 2.742 | SEQ ID NO: 160 |
| linc-MZF1-2 | 2.735 | SEQ ID NO: 161 |
| linc-CMPK1-3 | 2.663 | SEQ ID NO: 162 |
| linc-ARHGAP28-9 | 2.653 | SEQ ID NO: 163 |
| linc-NACC1 | 2.635 | SEQ ID NO: 164 |
| linc-BMS1-4 | 2.618 | SEQ ID NO: 165 |
| linc-TCP11L2-1 | 2.607 | SEQ ID NO: 166 |
| linc-CANX-1 | 2.564 | SEQ ID NO: 167 |
| linc-KCTD7-2 | 2.554 | SEQ ID NO: 168 |
| linc-TMEM105-2 | 2.538 | SEQ ID NO: 169 |
| linc-MRPS31 | 2.514 | SEQ ID NO: 170 |
| linc-RGL4-1 | 2.509 | SEQ ID NO: 171 |
| linc-METTL14-1 | 2.491 | SEQ ID NO: 172 |
| linc-NDUFB4-5 | 2.485 | SEQ ID NO: 173 |
| linc-ARF5-2 | 2.478 | SEQ ID NO: 174 |
| linc-NBPF15-1 | 2.467 | SEQ ID NO: 175 |
| linc-PHF10 | 2.454 | SEQ ID NO: 176 |
| linc-NADSYN1-1 | 2.450 | SEQ ID NO: 177 |
| linc-TMEM183B-1 | 2.434 | SEQ ID NO: 178 |
| linc-CALCOCO2-3 | 2.415 | SEQ ID NO: 179 |
| linc-BDH1-2 | 2.405 | SEQ ID NO: 180 |
| linc-ADAMTSL4 | 2.398 | SEQ ID NO: 181 |
| linc-RPS7-1 | 2.374 | SEQ ID NO: 182 |
| linc-ATP6V1C2-4 | 2.364 | SEQ ID NO: 183 |
| linc-FSCN2-1 | 2.340 | SEQ ID NO: 184 |
| linc-TUBGCP3-2 | 2.328 | SEQ ID NO: 185 |
| linc-HOXD1 | 2.314 | SEQ ID NO: 186 |
| linc-TGFBRAP1 | 2.301 | SEQ ID NO: 187 |
| linc-NOP14-3 | 2.273 | SEQ ID NO: 188 |
| linc-IER5L-2 | 2.253 | SEQ ID NO: 189 |
| linc-ASPRV1-1 | 2.230 | SEQ ID NO: 190 |
| linc-TPT1-2 | 2.206 | SEQ ID NO: 191 |
| linc-OAF-6 | 2.202 | SEQ ID NO: 192 |
| linc-COX5B-3 | 2.201 | SEQ ID NO: 193 |
| linc-ZBED1-4 | 2.200 | SEQ ID NO: 194 |
| linc-HIST1H2AI-1 | 2.182 | SEQ ID NO: 195 |
| linc-CALCOCO2-2 | 2.175 | SEQ ID NO: 196 |
| linc-ARF6-1 | 2.167 | SEQ ID NO: 197 |
| linc-MAP7-BP | 2.147 | SEQ ID NO: 198 |
| linc-LOC285033-4 | 2.146 | SEQ ID NO: 199 |
| linc-ZNF674 | 2.143 | SEQ ID NO: 200 |
| linc-HTR5A-1 | 2.130 | SEQ ID NO: 201 |
| linc-GPR179 | 2.072 | SEQ ID NO: 202 |
| linc-RPP40 | 2.069 | SEQ ID NO: 203 |
| linc-SATB2-2 | 2.067 | SEQ ID NO: 204 |
| linc-MUC20-2 | 2.062 | SEQ ID NO: 205 |
| linc-ZNF516-4 | 2.060 | SEQ ID NO: 206 |
| linc-STX17 | 2.037 | SEQ ID NO: 207 |
| linc-CDH6-7 | 2.035 | SEQ ID NO: 208 |
| linc-SERHL2-3 | 2.020 | SEQ ID NO: 209 |
| linc-OR4F16-4 | 2.004 | SEQ ID NO: 210 |

TABLE 3
DOWNREGULATED IN BREAST CANCER

| Gene_ID | lincRNA_ID | Fold_Change | SEQ ID NO. |
|---|---|---|---|
| XLOC_013732 | linc-GHRH | 0.499180468 | SEQ ID NO: 211 |
| XLOC_010622 | linc-VPS36-1 | 0.499124573 | SEQ ID NO: 212 |
| XLOC_013473 | linc-C20orf79 | 0.49850282 | SEQ ID NO: 213 |
| XLOC_006138 | linc-AUTS2-5 | 0.496339286 | SEQ ID NO: 214 |
| XLOC_011308 | linc-ISLR2-3 | 0.495656395 | SEQ ID NO: 215 |
| XLOC_000658 | linc-ZNF692-6 | 0.49203742 | SEQ ID NO: 216 |
| XLOC_012839 | linc-IER3IP1 | 0.491147796 | SEQ ID NO: 217 |
| XLOC_010020 | linc-MANSC1 | 0.489364178 | SEQ ID NO: 218 |
| XLOC_013868 | linc-CXADR-3 | 0.48782085 | SEQ ID NO: 219 |
| XLOC_012009 | linc-ZFHX3-4 | 0.485873656 | SEQ ID NO: 220 |
| XLOC_013350 | linc-ZNF404 | 0.484620723 | SEQ ID NO: 221 |
| XLOC_007996 | linc-FAAH2-2 | 0.483228361 | SEQ ID NO: 222 |
| XLOC_003471 | linc-CLRN2-1 | 0.483071468 | SEQ ID NO: 223 |
| XLOC_001343 | linc-ATP6V1C2-3 | 0.482227157 | SEQ ID NO: 224 |
| XLOC_003662 | linc-METTL14-2 | 0.481596879 | SEQ ID NO: 225 |
| XLOC_011819 | linc-MAP1LC3B-2 | 0.480798731 | SEQ ID NO: 226 |
| XLOC_009869 | linc-TCP11L2-1 | 0.479919818 | SEQ ID NO: 227 |
| XLOC_006037 | linc-GARS-1 | 0.478695771 | SEQ ID NO: 228 |
| XLOC_003860 | linc-NOP14-3 | 0.477680999 | SEQ ID NO: 229 |
| XLOC_004829 | linc-ANKRD55-6 | 0.477644486 | SEQ ID NO: 230 |
| XLOC_003734 | linc-ARFIP1-8 | 0.477497757 | SEQ ID NO: 231 |
| XLOC_012366 | linc-C17orf87 | 0.475047275 | SEQ ID NO: 232 |
| XLOC_012444 | linc-AMAC1 | 0.474129492 | SEQ ID NO: 233 |
| XLOC_012833 | linc-SYT4-1 | 0.47368454 | SEQ ID NO: 234 |
| XLOC_000734 | linc-HTR1D | 0.472227304 | SEQ ID NO: 235 |
| XLOC_014404 | linc-WNT7B-2 | 0.470874666 | SEQ ID NO: 236 |
| XLOC_009897 | linc-MAP1LC3B2-2 | 0.467638797 | SEQ ID NO: 237 |
| XLOC_011693 | linc-TP53TG3B-6 | 0.464429002 | SEQ ID NO: 238 |
| XLOC_012587 | linc-TMEM105-2 | 0.461761453 | SEQ ID NO: 239 |
| XLOC_005247 | linc-MICB | 0.461222628 | SEQ ID NO: 240 |
| XLOC_001560 | linc-PLGLB2 | 0.461144368 | SEQ ID NO: 241 |
| XLOC_000002 | linc-OR4F16-9 | 0.459563603 | SEQ ID NO: 242 |
| XLOC_007971 | linc-RBM10 | 0.457477759 | SEQ ID NO: 243 |
| XLOC_003782 | linc-KIAA1712-5 | 0.457345623 | SEQ ID NO: 244 |
| XLOC_003204 | linc-CBLB-6 | 0.453595662 | SEQ ID NO: 245 |
| XLOC_011117 | linc-ATG2B-2 | 0.453008229 | SEQ ID NO: 246 |
| XLOC_001952 | linc-ADI1 | 0.452755399 | SEQ ID NO: 247 |
| XLOC_008094 | linc-SPRY3-1 | 0.451474309 | SEQ ID NO: 248 |
| XLOC_011755 | linc-BEAN1 | 0.451290122 | SEQ ID NO: 249 |
| XLOC_008270 | linc-SHOX-5 | 0.449773022 | SEQ ID NO: 250 |
| XLOC_006031 | linc-WIPF3 | 0.449090252 | SEQ ID NO: 251 |
| XLOC_007907 | linc-SHOX-4 | 0.448976779 | SEQ ID NO: 252 |
| XLOC_001751 | linc-DCAF17-1 | 0.44797186 | SEQ ID NO: 253 |
| XLOC_000022 | linc-TNFRSF14 | 0.447491427 | SEQ ID NO: 254 |
| XLOC_010891 | linc-GPR65-6 | 0.442310612 | SEQ ID NO: 255 |
| XLOC_005940 | linc-PHF10 | 0.441180863 | SEQ ID NO: 256 |
| XLOC_000659 | linc-ZNF692-5 | 0.440733917 | SEQ ID NO: 257 |
| XLOC_008863 | linc-POLR3A-1 | 0.440296308 | SEQ ID NO: 258 |
| XLOC_006455 | linc-LOC389493-2 | 0.440180205 | SEQ ID NO: 259 |
| XLOC_012177 | linc-AP2B1-2 | 0.439517545 | SEQ ID NO: 260 |
| XLOC_008500 | linc-LRRTM3-3 | 0.439267969 | SEQ ID NO: 261 |
| XLOC_003130 | linc-SFMBT1 | 0.437923474 | SEQ ID NO: 262 |
| XLOC_010952 | linc-BTBD6-1 | 0.436794427 | SEQ ID NO: 263 |
| XLOC_001532 | linc-MTHFD2 | 0.436580148 | SEQ ID NO: 264 |
| XLOC_003111 | linc-PRSS42 | 0.43594778 | SEQ ID NO: 265 |
| XLOC_004484 | linc-RGMB-1 | 0.435374653 | SEQ ID NO: 266 |
| XLOC_008370 | linc-ITIH2-10 | 0.433890357 | SEQ ID NO: 267 |
| XLOC_005365 | linc-TPBG-3 | 0.433641735 | SEQ ID NO: 268 |
| XLOC_010097 | linc-TMEM194A | 0.426903381 | SEQ ID NO: 269 |
| XLOC_002710 | linc-FRG2C-3 | 0.424596849 | SEQ ID NO: 270 |
| XLOC_006188 | linc-ZKSCAN1-1 | 0.423272721 | SEQ ID NO: 271 |
| XLOC_005958 | linc-HEATR2-2 | 0.423084823 | SEQ ID NO: 272 |
| XLOC_006058 | linc-CDK13-1 | 0.421144709 | SEQ ID NO: 273 |
| XLOC_006293 | linc-GIMAP8-1 | 0.418403013 | SEQ ID NO: 274 |
| XLOC_009927 | linc-FAM101A-2 | 0.416533915 | SEQ ID NO: 275 |
| XLOC_009349 | linc-IFITM5 | 0.414748854 | SEQ ID NO: 276 |
| XLOC_008501 | linc-LRRTM3-2 | 0.413624264 | SEQ ID NO: 277 |
| XLOC_003663 | linc-METTL14-1 | 0.41231618 | SEQ ID NO: 278 |
| XLOC_002759 | linc-GTPBP8 | 0.412245121 | SEQ ID NO: 279 |
| XLOC_011201 | linc-KLF13-1 | 0.411702498 | SEQ ID NO: 280 |
| XLOC_013921 | linc-SLC5A3-2 | 0.40441622 | SEQ ID NO: 281 |
| XLOC_005816 | linc-BET3L | 0.401991105 | SEQ ID NO: 282 |
| XLOC_010734 | linc-TUBGCP3-2 | 0.401743633 | SEQ ID NO: 283 |
| XLOC_011769 | linc-CDH1 | 0.401444104 | SEQ ID NO: 284 |
| XLOC_010870 | linc-PTGR2 | 0.396154622 | SEQ ID NO: 285 |
| XLOC_010167 | linc-CDK17-4 | 0.394998914 | SEQ ID NO: 286 |
| XLOC_010377 | linc-COG3-3 | 0.394273047 | SEQ ID NO: 287 |
| XLOC_013929 | linc-CBR1-1 | 0.393741569 | SEQ ID NO: 288 |
| XLOC_002616 | linc-CCR8-3 | 0.393026799 | SEQ ID NO: 289 |
| XLOC_002323 | linc-LOC150786 | 0.392365681 | SEQ ID NO: 290 |
| XLOC_003839 | linc-FRG1-5 | 0.390890356 | SEQ ID NO: 291 |
| XLOC_011183 | linc-GABRA5-7 | 0.390854813 | SEQ ID NO: 292 |
| XLOC_008873 | linc-DYDC1-5 | 0.385000421 | SEQ ID NO: 293 |
| XLOC_001826 | linc-CREB1-1 | 0.383404985 | SEQ ID NO: 294 |
| XLOC_006752 | linc-LEPROTL1-7 | 0.383338011 | SEQ ID NO: 295 |
| XLOC_005587 | linc-C6orf145-3 | 0.382359817 | SEQ ID NO: 296 |
| XLOC_005214 | linc-HIST1H2AG-4 | 0.378694403 | SEQ ID NO: 297 |
| XLOC_011298 | linc-THSD4 | 0.37456428 | SEQ ID NO: 298 |
| XLOC_012393 | linc-HS3ST3A1-1 | 0.37414902 | SEQ ID NO: 299 |
| XLOC_010017 | linc-KLRC1 | 0.36789689 | SEQ ID NO: 300 |
| XLOC_013167 | linc-ZNF583-1 | 0.367389551 | SEQ ID NO: 301 |
| XLOC_012997 | linc-ZNF253-2 | 0.3670454 | SEQ ID NO: 302 |
| XLOC_005492 | linc-UTRN | 0.36526569 | SEQ ID NO: 303 |
| XLOC_001342 | linc-ATP6V1C2-4 | 0.364055202 | SEQ ID NO: 304 |
| XLOC_003872 | linc-GRPEL1-1 | 0.361963991 | SEQ ID NO: 305 |
| XLOC_001458 | linc-TTC7A-2 | 0.36182666 | SEQ ID NO: 306 |
| XLOC_010376 | linc-COG3-1 | 0.361425563 | SEQ ID NO: 307 |
| XLOC_010032 | linc-IFLTD1 | 0.360928317 | SEQ ID NO: 308 |
| XLOC_006296 | linc-GALNTL5-1 | 0.358756593 | SEQ ID NO: 309 |
| XLOC_006726 | linc-PCM1 | 0.358488671 | SEQ ID NO: 310 |
| XLOC_011309 | linc-ISLR2-2 | 0.353675742 | SEQ ID NO: 311 |
| XLOC_009493 | linc-DHCR7-2 | 0.352269235 | SEQ ID NO: 312 |
| XLOC_000678 | linc-HES5-2 | 0.350115239 | SEQ ID NO: 313 |
| XLOC_011248 | linc-USP8 | 0.348290046 | SEQ ID NO: 314 |
| XLOC_002952 | linc-VPS8-2 | 0.347817953 | SEQ ID NO: 315 |
| XLOC_014177 | linc-RGL4-1 | 0.345238799 | SEQ ID NO: 316 |
| XLOC_013042 | linc-CEBPG | 0.345038695 | SEQ ID NO: 317 |
| XLOC_007109 | linc-CRH-2 | 0.344933775 | SEQ ID NO: 318 |
| XLOC_000294 | linc-DR1 | 0.344051261 | SEQ ID NO: 319 |
| XLOC_007993 | linc-UBQLN2 | 0.343949716 | SEQ ID NO: 320 |
| XLOC_005332 | linc-MTRNR2L9-3 | 0.342479849 | SEQ ID NO: 321 |
| XLOC_001331 | linc-ID2-3 | 0.342191315 | SEQ ID NO: 322 |
| XLOC_000918 | linc-TMED5 | 0.340517434 | SEQ ID NO: 323 |
| XLOC_005753 | linc-RAB23-4 | 0.337208881 | SEQ ID NO: 324 |
| XLOC_010445 | linc-NDFIP2-1 | 0.336159171 | SEQ ID NO: 325 |
| XLOC_010791 | linc-ARHGAP5 | 0.335210921 | SEQ ID NO: 326 |
| XLOC_005127 | linc-WRNIP1-2 | 0.329920807 | SEQ ID NO: 327 |
| XLOC_007365 | linc-ANKRD20A1-14 | 0.329102587 | SEQ ID NO: 328 |
| XLOC_011280 | linc-TLN2-1 | 0.328482127 | SEQ ID NO: 329 |
| XLOC_002951 | linc-VPS8-3 | 0.323774938 | SEQ ID NO: 330 |
| XLOC_011790 | linc-CNTNAP4 | 0.322157444 | SEQ ID NO: 331 |
| XLOC_002726 | linc-CHMP2B-1 | 0.322080815 | SEQ ID NO: 332 |
| XLOC_013869 | linc-CXADR-2 | 0.315734934 | SEQ ID NO: 333 |
| XLOC_001575 | linc-LOC285033-5 | 0.314112195 | SEQ ID NO: 334 |
| XLOC_012618 | linc-ARHGAP28-2 | 0.313527431 | SEQ ID NO: 335 |
| XLOC_011592 | linc-RGMA-7 | 0.31075893 | SEQ ID NO: 336 |
| XLOC_008462 | linc-BMS1-3 | 0.310080067 | SEQ ID NO: 337 |
| XLOC_000677 | Iinc-C1orf86 | 0.308767935 | SEQ ID NO: 338 |
| XLOC_001809 | linc-CASP10-1 | 0.305027664 | SEQ ID NO: 339 |
| XLOC_003665 | linc-SYNPO2-2 | 0.304166908 | SEQ ID NO: 340 |
| XLOC_006242 | linc-FAM71F2-1 | 0.302572445 | SEQ ID NO: 341 |
| XLOC_006704 | linc-TNKS-3 | 0.302099578 | SEQ ID NO: 342 |
| XLOC_002353 | linc-MMADHC-3 | 0.301706409 | SEQ ID NO: 343 |
| XLOC_006753 | linc-LEPROTL1-6 | 0.300180236 | SEQ ID NO: 344 |
| XLOC_011480 | linc-BCL2L10 | 0.300133861 | SEQ ID NO: 345 |
| XLOC_010967 | linc-OR5AU1 | 0.299893673 | SEQ ID NO: 346 |
| XLOC_005361 | linc-SH3BGRL2-1 | 0.299587053 | SEQ ID NO: 347 |
| XLOC_002209 | linc-TRIM43B-2 | 0.298151464 | SEQ ID NO: 348 |
| XLOC_007808 | linc-ALG2-5 | 0.29496598 | SEQ ID NO: 349 |
| XLOC_006721 | linc-C8orf79-2 | 0.294495313 | SEQ ID NO: 350 |
| XLOC_013559 | linc-CEBPB-1 | 0.293224 | SEQ ID NO: 351 |
| XLOC_005052 | linc-RPS14 | 0.292874422 | SEQ ID NO: 352 |
| XLOC_008916 | linc-RRP12 | 0.291314841 | SEQ ID NO: 353 |
| XLOC_002048 | linc-FAM98A-3 | 0.290100326 | SEQ ID NO: 354 |
| XLOC_003422 | linc-TACC3 | 0.288364688 | SEQ ID NO: 355 |
| XLOC_012773 | linc-MPPE1-4 | 0.287516341 | SEQ ID NO: 356 |
| XLOC_013631 | linc-IDH3B-1 | 0.286798877 | SEQ ID NO: 357 |
| XLOC_006719 | linc-C8orf79-4 | 0.282627148 | SEQ ID NO: 358 |
| XLOC_006994 | linc-DEFB105B-3 | 0.28231064 | SEQ ID NO: 359 |
| XLOC_005452 | linc-HSF2-1 | 0.281989523 | SEQ ID NO: 360 |
| XLOC_001401 | linc-EPT1 | 0.279663655 | SEQ ID NO: 361 |
| XLOC_010754 | linc-RPGRIP1-3 | 0.274995392 | SEQ ID NO: 362 |
| XLOC_002997 | linc-MUC20-1 | 0.273721122 | SEQ ID NO: 363 |
| XLOC_006507 | linc-MAGI2-3 | 0.273664097 | SEQ ID NO: 364 |
| XLOC_001309 | linc-SNTG2-3 | 0.270862516 | SEQ ID NO: 365 |
| XLOC_006993 | linc-DEFB105B-2 | 0.268784648 | SEQ ID NO: 366 |
| XLOC_005924 | linc-TCP10-4 | 0.265082165 | SEQ ID NO: 367 |
| XLOC_009813 | linc-NAV3-1 | 0.263629068 | SEQ ID NO: 368 |
| XLOC_014111 | linc-CSTB-6 | 0.263146049 | SEQ ID NO: 369 |
| XLOC_008617 | linc-TCF7L2-3 | 0.261553826 | SEQ ID NO: 370 |
| XLOC_012083 | linc-SPNS3 | 0.258809064 | SEQ ID NO: 371 |
| XLOC_011350 | linc-FURIN | 0.258505692 | SEQ ID NO: 372 |
| XLOC_009765 | linc-HNRNPA1-3 | 0.254982021 | SEQ ID NO: 373 |
| XLOC_000746 | linc-C1orf63 | 0.254843625 | SEQ ID NO: 374 |
| XLOC_001415 | linc-SPAST-2 | 0.253244998 | SEQ ID NO: 375 |
| XLOC_007615 | linc-EGFL7-1 | 0.247945267 | SEQ ID NO: 376 |
| XLOC_005939 | linc-THBS2-3 | 0.247150699 | SEQ ID NO: 377 |
| XLOC_007347 | linc-FRMPD1 | 0.244838556 | SEQ ID NO: 378 |
| XLOC_014121 | linc-ITGB2-3 | 0.241913784 | SEQ ID NO: 379 |
| XLOC_011104 | linc-TRIP11 | 0.239932263 | SEQ ID NO: 380 |
| XLOC_004501 | linc-FER-1 | 0.23938625 | SEQ ID NO: 381 |
| XLOC_010263 | linc-TMEM132D-1 | 0.237899784 | SEQ ID NO: 382 |
| XLOC_011081 | linc-C14orf4-2 | 0.237568632 | SEQ ID NO: 383 |
| XLOC_001208 | linc-GPATCH2-9 | 0.2321516 | SEQ ID NO: 384 |
| XLOC_012726 | linc-ZNF236-4 | 0.229945164 | SEQ ID NO: 385 |
| XLOC_000889 | linc-TTLL7-2 | 0.22550242 | SEQ ID NO: 386 |
| XLOC_011193 | linc-APBA2-3 | 0.223522184 | SEQ ID NO: 387 |
| XLOC_008795 | linc-ZNF32-5 | 0.220751156 | SEQ ID NO: 388 |
| XLOC_011388 | linc-ALDH1A3-1 | 0.216685456 | SEQ ID NO: 389 |
| XLOC_000909 | linc-GBP5-2 | 0.214717471 | SEQ ID NO: 390 |
| XLOC_006178 | linc-DYNC1I1-2 | 0.210154936 | SEQ ID NO: 391 |
| XLOC_011368 | linc-NR2F2-3 | 0.209672378 | SEQ ID NO: 392 |
| XLOC_005764 | linc-B3GAT2-4 | 0.208500606 | SEQ ID NO: 393 |
| XLOC_009255 | linc-CEP57-3 | 0.198606717 | SEQ ID NO: 394 |
| XLOC_011880 | linc-CPPED1-3 | 0.197968745 | SEQ ID NO: 395 |
| XLOC_008489 | linc-PHYHIPL | 0.195626262 | SEQ ID NO: 396 |
| XLOC_007438 | linc-C9orf170-1 | 0.187831194 | SEQ ID NO: 397 |
| XLOC_012171 | linc-SPACA3 | 0.184937122 | SEQ ID NO: 398 |
| XLOC_011334 | linc-FAM103A1 | 0.181631176 | SEQ ID NO: 399 |
| XLOC_001640 | linc-INHBB | 0.177266105 | SEQ ID NO: 400 |
| XLOC_007377 | linc-ANKRD20A1-2 | 0.174463897 | SEQ ID NO: 401 |
| XLOC_001061 | linc-CRP | 0.173769194 | SEQ ID NO: 402 |
| XLOC_003449 | linc-CPEB2-16 | 0.17280083 | SEQ ID NO: 403 |
| XLOC_000021 | linc-PLCH2 | 0.16678836 | SEQ ID NO: 404 |
| XLOC_012112 | linc-SHISA6-1 | 0.162855407 | SEQ ID NO: 405 |
| XLOC_003795 | linc-ODZ3-5 | 0.15538216 | SEQ ID NO: 406 |
| XLOC_001035 | linc-C1orf43-2 | 0.155203665 | SEQ ID NO: 407 |
| XLOC_005221 | linc-HIST1H2AI-1 | 0.152407981 | SEQ ID NO: 408 |
| XLOC_008735 | linc-BEND7-1 | 0.150345336 | SEQ ID NO: 409 |
| XLOC_009356 | linc-CTSD-3 | 0.146136121 | SEQ ID NO: 410 |
| XLOC_002035 | linc-RBKS-1 | 0.145821566 | SEQ ID NO: 411 |
| XLOC_000595 | linc-PRSS38 | 0.143439674 | SEQ ID NO: 412 |
| XLOC_002070 | linc-HAAO-6 | 0.14205683 | SEQ ID NO: 413 |
| XLOC_000179 | linc-SLC5A9-4 | 0.141891907 | SEQ ID NO: 414 |
| XLOC_002344 | linc-ZEB2-7 | 0.136925129 | SEQ ID NO: 415 |
| XLOC_007701 | linc-FAM75A6-7 | 0.136246423 | SEQ ID NO: 416 |
| XLOC_010693 | linc-DCT-2 | 0.133372302 | SEQ ID NO: 417 |
| XLOC_002368 | linc-LY75 | 0.131903707 | SEQ ID NO: 418 |
| XLOC_009378 | linc-DKK3-3 | 0.131310221 | SEQ ID NO: 419 |
| XLOC_009361 | linc-TRPM5 | 0.130680254 | SEQ ID NO: 420 |
| XLOC_010336 | linc-USPL1-1 | 0.127297353 | SEQ ID NO: 421 |
| XLOC_010592 | linc-TPT1-2 | 0.115058273 | SEQ ID NO: 422 |
| XLOC_007880 | linc-OBP2B | 0.106853371 | SEQ ID NO: 423 |
| XLOC_004252 | linc-C5orf38-4 | 0.106063415 | SEQ ID NO: 424 |
| XLOC_000682 | linc-MMEL1-3 | 0.105313513 | SEQ ID NO: 425 |
| XLOC_006294 | linc-GALNTL5-3 | 0.10516268 | SEQ ID NO: 426 |
| XLOC_008955 | linc-ZDHHC6-2 | 0.094186466 | SEQ ID NO: 427 |
| XLOC_004250 | linc-C5orf38-5 | 0.092365339 | SEQ ID NO: 428 |
| XLOC_002067 | linc-HAAO-4 | 0.09050779 | SEQ ID NO: 429 |
| XLOC_008151 | linc-CXorf36-3 | 0.088444922 | SEQ ID NO: 430 |
| XLOC_000105 | linc-WDTC1 | 0.088191677 | SEQ ID NO: 431 |
| XLOC_005991 | linc-LOC100129335-2 | 0.080936735 | SEQ ID NO: 432 |
| XLOC_009261 | linc-DYNC2H1-4 | 0.080797382 | SEQ ID NO: 433 |
| XLOC_013305 | linc-PEPD-1 | 0.080031439 | SEQ ID NO: 434 |
| XLOC_005827 | linc-HDDC2-4 | 0.077438115 | SEQ ID NO: 435 |
| XLOC_010593 | linc-TPT1-1 | 0.072867073 | SEQ ID NO: 436 |
| XLOC_013900 | linc-USP16-5 | 0.072684312 | SEQ ID NO: 437 |
| XLOC_014237 | linc-PICK1 | 0.065587113 | SEQ ID NO: 438 |
| XLOC_008224 | linc-RHOXF1-3 | 0.061433191 | SEQ ID NO: 439 |
| XLOC_004800 | linc-OXCT1-1 | 0.060013638 | SEQ ID NO: 440 |
| XLOC_005088 | linc-BOD1-2 | 0.059687797 | SEQ ID NO: 441 |
| XLOC_004598 | linc-ARHGEF37-2 | 0.057257232 | SEQ ID NO: 442 |
| XLOC_004517 | linc-AQPEP | 0.048804254 | SEQ ID NO: 443 |
| XLOC_004090 | linc-PABPC4L-1 | 0.046101682 | SEQ ID NO: 444 |
| XLOC_011816 | linc-MAP1LC3B-5 | 0.044516113 | SEQ ID NO: 445 |
| XLOC_011944 | linc-TOX3-2 | 0.044321378 | SEQ ID NO: 446 |
| XLOC_010826 | linc-FRMD6-2 | 0.039242318 | SEQ ID NO: 447 |
| XLOC_005629 | linc-DEK-6 | 0.030014166 | SEQ ID NO: 448 |
| XLOC_013795 | linc-ZFP64-5 | 0.025959582 | SEQ ID NO: 449 |
| XLOC_000207 | linc-PRKAA2-8 | 0.02425015 | SEQ ID NO: 450 |
| XLOC_012564 | linc-ABCA5-7 | 0.023910303 | SEQ ID NO: 451 |
| XLOC_007725 | linc-PRKACG-2 | 0.0229167 | SEQ ID NO: 452 |

TABLE 4
UPREGULATED IN BREAST TUMORS

| Gene_ID | lincRNA_ID | Fold_Change | SEQ ID NO. |
|---|---|---|---|
| XLOC_001851 | linc-TMEM169-3 | 58.83955766 | SEQ ID NO: 453 |
| XLOC_012538 | linc-HEATR6-2 | 15.36968877 | SEQ ID NO: 454 |
| XLOC_002866 | linc-TM4SF4-2 | 14.32014203 | SEQ ID NO: 455 |
| XLOC_005936 | linc-DACT2-3 | 13.10115726 | SEQ ID NO: 456 |
| XLOC_012925 | linc-SAFB-2 | 9.850864322 | SEQ ID NO: 457 |
| XLOC_013207 | linc-PTPRS-2 | 8.815825271 | SEQ ID NO: 458 |
| XLOC_012193 | linc-ZPBP2 | 5.568541345 | SEQ ID NO: 459 |
| XLOC_002221 | linc-MGAT4A | 5.490873802 | SEQ ID NO: 460 |
| XLOC_007053 | linc-DUSP26-5 | 5.08426453 | SEQ ID NO: 461 |
| XLOC_012057 | linc-CA5A-2 | 4.983267364 | SEQ ID NO: 462 |
| XLOC_001625 | linc-MERTK-2 | 4.380662896 | SEQ ID NO: 463 |
| XLOC_012568 | linc-ABCA5-3 | 4.334677593 | SEQ ID NO: 464 |
| XLOC_000160 | linc-GUCA2B | 4.214663722 | SEQ ID NO: 465 |
| XLOC_001763 | linc-HOXD1 | 4.149028136 | SEQ ID NO: 466 |
| XLOC_010998 | linc-FOXA1-2 | 3.8345659 | SEQ ID NO: 467 |
| XLOC_001257 | linc-EGLN1-2 | 3.760908853 | SEQ ID NO: 468 |
| XLOC_011294 | linc-LRRC49-4 | 3.630447216 | SEQ ID NO: 469 |
| XLOC_001942 | linc-TMEM18-13 | 3.488354904 | SEQ ID NO: 470 |
| XLOC_013301 | linc-ANKRD27 | 3.457454839 | SEQ ID NO: 471 |
| XLOC_012754 | linc-LAMA1-5 | 3.444137534 | SEQ ID NO: 472 |
| XLOC_000527 | linc-TMEM183B-1 | 3.095113275 | SEQ ID NO: 473 |
| XLOC_003932 | linc-UGDH | 3.009349439 | SEQ ID NO: 474 |
| XLOC_011858 | linc-PKMYT1 | 2.948945954 | SEQ ID NO: 475 |
| XLOC_012192 | linc-PPP1R1B | 2.90336726 | SEQ ID NO: 476 |
| XLOC_013534 | linc-WFDC2-2 | 2.851108866 | SEQ ID NO: 477 |
| XLOC_003077 | linc-DYNC1LI1-2 | 2.777741675 | SEQ ID NO: 478 |
| XLOC_010236 | linc-DHX37-22 | 2.764514331 | SEQ ID NO: 479 |
| XLOC_009299 | linc-OAF-6 | 2.718723501 | SEQ ID NO: 480 |
| XLOC_014418 | linc-ODF3B | 2.624478464 | SEQ ID NO: 481 |
| XLOC_012611 | linc-ARHGAP28-9 | 2.361504587 | SEQ ID NO: 482 |
| XLOC_007054 | linc-DUSP26-6 | 2.336774622 | SEQ ID NO: 483 |
| XLOC_002408 | linc-EVX2-8 | 2.277839612 | SEQ ID NO: 484 |
| XLOC_012503 | linc-COPZ2 | 2.21559804 | SEQ ID NO: 485 |
| XLOC_011034 | linc-DLGAP5-1 | 2.188339707 | SEQ ID NO: 486 |
| XLOC_004445 | linc-XRCC4-3 | 2.152494357 | SEQ ID NO: 487 |
| XLOC_010709 | linc-ZIC5 | 2.129794315 | SEQ ID NO: 488 |
| XLOC_008724 | linc-KIN-5 | 2.114328259 | SEQ ID NO: 489 |
| XLOC_012975 | linc-NACC1 | 2.10115787 | SEQ ID NO: 490 |
| XLOC_002133 | linc-SERTAD2-4 | 2.046833149 | SEQ ID NO: 491 |
| XLOC_012342 | linc-ASPSCR1 | 2.022831858 | SEQ ID NO: 492 |
| XLOC_003441 | linc-KIAA0232 | 2.007391308 | SEQ ID NO: 493 |

TABLE 5
DOWNREGULATED IN BOTH
COLON AND BREAST TUMORS

| lincRNA_ID | SEQ ID NO. |
|---|---|
| linc-AP2B1-2 | SEQ ID NO: 494 |
| linc-UTRN | SEQ ID NO: 495 |
| linc-GPR65-6 | SEQ ID NO: 496 |
| linc-EGFL7-1 | SEQ ID NO: 497 |
| linc-ZNF404 | SEQ ID NO: 498 |
| linc-FRG1-5 | SEQ ID NO: 499 |
| linc-BCL2L10 | SEQ ID NO: 500 |
| linc-GIMAP8-1 | SEQ ID NO: 501 |
| linc-MAGI2-3 | SEQ ID NO: 502 |
| linc-DEFB105B-2 | SEQ ID NO: 503 |
| linc-C1orf43-2 | SEQ ID NO: 504 |
| linc-RGMA-7 | SEQ ID NO: 505 |
| linc-SHISA6-1 | SEQ ID NO: 506 |
| linc-SYT4-1 | SEQ ID NO: 507 |
| linc-GRPEL1-1 | SEQ ID NO: 508 |
| linc-ZNF583-1 | SEQ ID NO: 509 |
| linc-ZEB2-7 | SEQ ID NO: 510 |
| linc-GPATCH2-9 | SEQ ID NO: 511 |
| linc-ARHGEF37-2 | SEQ ID NO: 512 |
| linc-GTPBP8 | SEQ ID NO: 513 |

TABLE 6
UPREGULATED IN BOTH COLON AND BREAST TUMORS

| lincRNA_ID | SEQ ID NO. |
|---|---|
| linc-DUSP26-6 | SEQ ID NO: 514 |
| linc-ASPSCR1 | SEQ ID NO: 515 |
| linc-SERTAD2-4 | SEQ ID NO: 516 |
| linc-ARHGAP28-9 | SEQ ID NO: 517 |
| linc-NACC1 | SEQ ID NO: 518 |
| linc-TMEM183B-1 | SEQ ID NO: 519 |
| linc-HOXD1 | SEQ ID NO: 520 |
| linc-OAF-6 | SEQ ID NO: 521 |

In some embodiments, cancer in a subject can be treated by administering an agent to cancer cells of the subject at an amount effective to modulate the level of DNMT1-associated RNA and/or the interaction of DNMT1-associated RNA and DNMT1 in the cancer cells. In one example, the cancer can be selected from the group consisting of breast cancer and colon cancer. In other embodiments, the DNMT1-associated RNA can be DNMT1-associated long non-coding RNA.

In some embodiments, the agent administered to the cancer cells can be effective to decrease, reduce or downregulate the level of DNMT1-associated RNA that is over-expressed or upregulated in the cancer cells compared to normal cells. As used herein, the term “downregulate”, or “reduce”, means that the level of DNMT1-associated RNA molecules or equivalent RNA is reduced below that observed in comparative normal cells. The DNMT1-associated RNA is down-regulated when expression of the DNMT1-associated RNA molecules is reduced at least 10%, at least about 20%, at least about 30%, at least about 50%, or at least about 75% relative to a corresponding non-modulated control. Thus, in some embodiments, the agent can be an inhibitor (e.g., antagonist) of DNMT1-associated RNA that is upregulated or over expressed in the cancer cells compared to normal cells.

In one example, the DNMT-1 associated RNA that is over expressed or upregulated can include at least one of linc-GATA5-1, linc-FAM84B-9, linc-OR10H4, linc-DUSP26-6, linc-CCDC40-1, linc-CSPP1, linc-ASPSCR1, linc-U2AF1-5, linc-BEAN1, linc-EFR3A-7, linc-SLC25A45-5, linc-SEMA3A, linc-CXXC4-1, linc-EFR3A-4, linc-JAKMIP3-3, linc-KIAA1755-4, linc-EPHB4, linc-GAD1-1, linc-IGFBP2-3, linc-CCDC122-4, linc-NADSYN1-2, linc-DUSP26-1, linc-EFR3A-5, linc-TCF20, linc-RSPH1-1, linc-DUXA-2, linc-RTEL1, linc-INO80, linc-UBE3C-2, linc-STIM2-1, linc-VEZF1, linc-GPR183-2, linc-WHAMM-1, linc-FRMPD1, linc-MIB2, linc-SERTAD2-4, linc-HAAO-4, linc-CDH5-3, linc-NDUFAF2-3, linc-PPM1J, linc-LY6H, linc-MKLN1-2, linc-SERPIND1, linc-TCP10-5, linc-PPIAL4F-1, linc-BIRC7-3, linc-S100B-2, linc-C1QTNF9B, linc-PXN, linc-SRL, linc-ZNF692-6, linc-BDH1-3, linc-RALGAPB, linc-MYOD1, linc-OR4F16-9, linc-MUC20-3, linc-BTBD6-1, linc-CDK13-1, linc-ZNF8-2, linc-HIST1H2AI-2, linc-OR7C2-1, linc-MZF1-2, linc-CMPK1-3, linc-ARHGAP28-9, linc-NACC1, linc-BMS1-4, linc-TCP11L2-1, linc-CANX-1, linc-KCTD7-2, linc-TMEM105-2, linc-MRPS31, linc-RGL4-1, linc-METTL14-1, linc-NDUFB4-5, linc-ARF5-2, linc-NBPF15-1, linc-PHF10, linc-NADSYN1-1, linc-TMEM183B-1, linc-CALCOCO2-3, linc-BDH1-2, linc-ADAMTSL4, linc-RPS7-1, linc-ATP6V1C2-4, linc-FSCN2-1, linc-TUBGCP3-2, linc-HOXD1, linc-TGFBRAP1, linc-NOP14-3, linc-IER5L-2, linc-ASPRV1-1, linc-TPT1-2, linc-OAF-6, linc-COX5B-3, linc-ZBED1-4, linc-HIST1H2AI-1, linc-CALCOCO2-2, linc-ARF6-1, linc-MAP7-BP, linc-LOC285033-4, linc-ZNF674, linc-HTR5A-1, linc-GPR179, linc-RPP40, linc-SATB2-2, linc-MUC20-2, linc-ZNF516-4, linc-STX17, linc-CDH6-7, linc-SERHL2-3, or linc-OR4F16-4.

In another example, the DNMT-1 associated RNA that is over expressed can include at least one of linc-TMEM169-3, linc-HEATR6-2, linc-TM4SF4-2, linc-DACT2-3, linc-SAFB-2, linc-PTPRS-2, linc-ZPBP2, linc-MGAT4A, linc-DUSP26-5, linc-CA5A-2, linc-MERTK-2, linc-ABCA5-3, linc-GUCA2B, linc-HOXD1, linc-FOXA1-2, linc-EGLN1-2, linc-LRRC49-4, linc-TMEM18-13, linc-ANKRD27, linc-LAMA1-5, linc-TMEM183B-1, linc-UGDH, linc-PKMYT1, linc-PPP1R1B, linc-WFDC2-2, linc-DYNC1LI1-2, linc-DHX37-22, linc-OAF-6, linc-ODF3B, linc-ARHGAP28-9, linc-DUSP26-6, linc-EVX2-8, linc-COPZ2, linc-DLGAP5-1, linc-XRCC4-3, linc-ZIC5, linc-KIN-5, linc-NACC1, linc-SERTAD2-4, linc-ASPSCR1, or linc-KIAA0232.

In yet another example, the DNMT-1 associated RNA can include at least one of linc-DUSP26-6, linc-ASPSCR1, linc-SERTAD2-4, linc-ARHGAP28-9, linc-NACC1, linc-TMEM183B-1, linc-HOXD1, or linc-OAF-6.

An inhibitor of DNMT1-associated RNA, which is upregulated or over expressed in cancer cells compared to normal cells, can include any agent that inhibits or reduces DNMT1-associated RNA expression or function. Agents that inhibit or reduce DNMT1-associated RNA expression or function can be any type of entity, for example, chemicals, nucleic acid sequences, nucleic acid analogues, proteins, peptides or fragments thereof. In some embodiments, the agent is any chemical, entity or moiety, including without limitation, synthetic and naturally-occurring non-proteinaceous entities. In certain embodiments the agent is a small molecule having a chemical moiety.

In some embodiments, agents that inhibit or reduce DNMT1-associated RNA expression or function are nucleic acids. Nucleic acid inhibitors of DNMT1-associated RNA expression or function include, for example, RNA interference (RNAi) molecules or constructs, such as siRNA, dsRNA, stRNA, shRNA, microRNA and modified versions thereof, where the RNA interference molecule silences the expression or function of the DNMT1-associated RNA. The RNAi molecule of DNMT1-associated RNA can have nucleic acid sequence that is substantially complementary to a portion of at least one DNMT1-associated RNA that is upregulated in the cancer cells. For example, the RNAi molecule of DNMT1-associated RNA can have nucleic acid sequence that is substantially complementary to a portion of at least one DNMT1-associated RNA, which is listed in Tables 2, 4, and 6.

In some embodiments single-stranded RNA (ssRNA), a form of RNA endogenously found in eukaryotic cells can be used to form an RNAi molecule. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

RNA interference (RNAi) provides a powerful approach for inhibiting the expression of selected target RNAs. RNAi uses small interfering RNA (siRNA) duplexes that target the RNA for selective degradation. siRNA-dependent post-transcriptional silencing of gene expression involves cutting the target messenger RNA molecule at a site guided by the siRNA.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or level of the target gene as compared to a situation wherein no RNA interference has been induced. The decrease can be of at least about 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” refers to an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs.

The siRNA targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. (Id.) 15, or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompass chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Other siRNAs useful for targeting DNMT1-associated RNA expression or function can be readily designed and tested. Accordingly, siRNAs useful for the methods described herein include siRNA molecules of about 15 to about 40 or about 15 to about 28 nucleotides in length. In some embodiments, the DNMT1-associated RNA targeting siRNA molecules can have a length of about 25 to about 29 nucleotides. In other embodiments, the DNMT1-associated RNA targeting siRNA molecules have a length of about 27, 28, 29, or 30 nucleotides. The DNMT1-associated RNA targeting siRNA molecules can also comprise a 3′ hydroxyl group. The DNMT1-associated RNA targeting siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In some embodiments, the siRNA or modified siRNA, such as gene silencing RNAi agents, and/or gene activating RNAi agents are delivered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting, for example, the DNMT1-associated RNA, to inhibit its function and/or expression. In one embodiment, the vector can be a regulatable vector, such as tetracycline inducible vector.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods described herein.

Other strategies for delivery of the RNA interfering agents, e.g., the siRNAs or shRNAs used in the methods described herein, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a episomal vector provides a means of maintaining the antagonist nucleic acid molecule in the subject in high copy number extra chromosomal DNA thereby eliminating potential effects of chromosomal integration.

RNA interference molecules and nucleic acid inhibitors used in the methods as disclosed herein can be produced using any known techniques, such as direct chemical synthesis, through processing of longer double stranded RNAs by exposure to recombinant Dicer protein or Drosophila embryo lysates, through an in vitro system derived from S2 cells, using phage RNA polymerase, RNA-dependant RNA polymerase, and DNA based vectors. Use of cell lysates or in vitro processing can further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.)

In one embodiment, an inhibitor of DNMT1-associated RNA function and/or its expression can be obtained synthetically, for example, by chemically synthesizing a nucleic acid by any method of synthesis known to the skilled artisan. A synthesized nucleic acid inhibitor of DNMT1-associated RNA function and/or its expression can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

Synthetic siRNA molecules, including shRNA molecules, can also easily be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but are not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell. 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

Methods of delivering RNAi agents, e.g., a siRNA, or vectors containing an RNAi agent, to the target cells (e.g., colon cancer cells, breast cancer cells, or other desired target cells) are well known to persons of ordinary skill in the art. In some embodiments, a RNAi agent inhibitor of DNMT1-associated RNA function and/or its expression can be administered to a subject by injection of a composition containing the RNA interfering agent, e.g., an siRNA, or directly contacting the cell with a composition comprising an RNAi agent, e.g., an siRNA. In another embodiment, RNAi agents, e.g., a siRNA can be injected directly into any blood vessel, such as vein, artery, venule or arteriole, via, e.g., hydrodynamic injection or catheterization.

Administration can be by a single injection or by two or more injections. In some embodiments, a RNAi agent is delivered in a pharmaceutically acceptable carrier. A gene silencing-RNAi agent which inhibits DNMT1-associated RNA function and/or its expression can also be administered in combination with other pharmaceutical agents which are used to treat or prevent cancer.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNAi effectively into cells. In some embodiments, a siRNA or RNAi binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

In some embodiments, a viral-mediated delivery mechanism can also be employed to deliver siRNAs, e.g., siRNAs (e.g., gene silencing-RNAi agents) which inhibits DNMT1-associated RNA function and/or its expression to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501).

The dose of the particular RNAi agent will be in an amount necessary to effect RNA interference, e.g., gene silencing RNAi which inhibits DNMT1-associated RNA function and/or its expression leading to reduction of DNMT1-associated RNA level.

In other embodiments, an agent that modulates the level of DNMT1-associated RNA and/or the interaction of DNMT1-associated RNA and DNMT1 in the cancer cells of the subject can be an agent that increases, enhances or upregulates the level of DNMT1-associated RNA, which is under-expressed or downregulated in the cancer cells compared to normal cells. The agent can include, for example, a nucleic acid encoding the under expressed or downregulated DNMT1-associated RNA in the cancer cells.

In one example, the DNMT1-associated RNA that is under expressed or downregulated in the cancer cells can include at least one of linc-SMAD3 (DACOR1), linc-ANXA8L2-2, linc-TRAK1, linc-AP2B1-2, linc-UTRN, linc-TBX18, linc-GPR65-6, linc-STIL-2, linc-ENPP6-2, linc-GABRA5-6, linc-MRPS18C, linc-EGFL7-1, linc-KLHL31-2, linc-PSMA8-3, linc-ZNF404, linc-HMGB2, linc-OAF-4, linc-FRG1-5, linc-HIST1H3A, linc-TMEM56-3, linc-DBT-3, linc-GNAI1-2, linc-BCL2L10, linc-EPHA6-1, linc-PLDN, linc-GABRA5-5, linc-ACO1-2, linc-NEDD4L-1, linc-MTRNR2L1-2, linc-FAM155B, linc-GIMAP8-1, linc-MAGI2-3, linc-DHX37-17, linc-KLF6-3, linc-RAP1GAP2-1, linc-TMPRSS2-2, linc-C10orf57-3, linc-GPR157-3, linc-LAMA4-2, linc-STIM1, linc-RFC2-2, linc-MRGPRF-1, linc-DEFB105B-2, linc-CTDSP2-1, linc-PRPS1L1, linc-SLC19A1-4, linc-Clorf43-2, linc-COX4NB-8, linc-HES1-3, linc-FIGNL1, linc-OAF-2, linc-COX4NB-9, linc-FBXL5-2, linc-TMEM220-2, linc-KCNMB2-5, linc-KIAA0141, linc-DHX37-19, linc-RGMA-7, linc-ID2-1, linc-SHISA6-1, linc-SYT4-1, linc-TRIML2-5, linc-DHFRL1-4, linc-RGS9-1, linc-ODF2L, linc-SLC22A16, linc-ZPBP2, linc-AGMAT-3, linc-MT1B, linc-GRPEL1-1, linc-PFDN4-2, linc-OPRK1-4, linc-ZNF583-1, linc-PFDN4-3, linc-SAMSN1-3, linc-USP3-1, linc-SHISA6-2, linc-ADAM29-3, linc-ZEB2-7, linc-MLL5, linc-FOXF1-3, linc-BTBD3-3, linc-GPATCH2-9, linc-ARHGEF37-2, linc-KLF6-2, linc-CLMN-1, linc-FOXG1-4, linc-TAAR9-1, linc-GTPBP8, linc-ADAR, linc-SAFB-2, linc-CXorf49B-2, linc-SLCO2A1-1, linc-PTPRS-2, linc-EPCAM, linc-LPHN2-1, linc-AMN1, linc-FAM55D, linc-FAM75A6-4, or linc-PHOX2B-2.

In another example, the DNMT1-associated RNA that is under expressed in the cancer cells can include at least one of linc-GHRH, linc-VPS36-1, linc-C20orf79, linc-AUTS2-5, linc-ISLR2-3, linc-ZNF692-6, linc-IER3IP1, linc-MANSC1, linc-CXADR-3, linc-ZFHX3-4, linc-ZNF404, linc-FAAH2-2, linc-CLRN2-1, linc-ATP6V1C2-3, linc-METTL14-2, linc-MAP1LC3B-2, linc-TCP11L2-1, linc-GARS-1, linc-NOP14-3, linc-ANKRD55-6, linc-ARFIP1-8, linc-C17orf87, linc-AMAC1, linc-SYT4-1, linc-HTR1D, linc-WNT7B-2, linc-MAP1LC3B2-2, linc-TP53TG3B-6, linc-TMEM105-2, linc-MICB, linc-PLGLB2, linc-OR4F16-9, linc-RBM10, linc-KIAA1712-5, linc-CBLB-6, linc-ATG2B-2, linc-ADI1, linc-SPRY3-1, linc-BEAN1, linc-SHOX-5, linc-WIPF3, linc-SHOX-4, linc-DCAF17-1, linc-TNFRSF14, linc-GPR65-6, linc-PHF10, linc-ZNF692-5, linc-POLR3A-1, linc-LOC389493-2, linc-AP2B1-2, linc-LRRTM3-3, linc-SFMBT1, linc-BTBD6-1, linc-MTHFD2, linc-PRSS42, linc-RGMB-1, linc-ITIH2-10, linc-TPBG-3, linc-TMEM194A, linc-FRG2C-3, linc-ZKSCAN1-1, linc-HEATR2-2, linc-CDK13-1, linc-GIMAP8-1, linc-FAM101A-2, linc-IFITM5, linc-LRRTM3-2, linc-METTL14-1, linc-GTPBP8, linc-KLF13-1, linc-SLC5A3-2, linc-BET3L, linc-TUBGCP3-2, linc-CDH1, linc-PTGR2, linc-CDK17-4, linc-COGS-3, linc-CBR1-1, linc-CCR8-3, linc-LOC150786, linc-FRG1-5, linc-GABRA5-7, linc-DYDC1-5, linc-CREB1-1, linc-LEPROTL1-7, linc-C6orf145-3, linc-HIST1H2AG-4, linc-THSD4, linc-HS3ST3A1-1, linc-KLRC1, linc-ZNF583-1, linc-ZNF253-2, linc-UTRN, linc-ATP6V1C2-4, linc-GRPEL1-1, linc-TTC7A-2, linc-COGS-1, linc-IFLTD1, linc-GALNTL5-1, linc-PCM1, linc-ISLR2-2, linc-DHCR7-2, linc-HES5-2, linc-USP8, linc-VPS8-2, linc-RGL4-1, linc-CEBPG, linc-CRH-2, linc-DR1, linc-UBQLN2, linc-MTRNR2L9-3, linc-ID2-3, linc-TMED5, linc-RAB23-4, linc-NDFIP2-1, linc-ARHGAP5, linc-WRNIP1-2, linc-ANKRD20A1-14, linc-TLN2-1, linc-VPS8-3, linc-CNTNAP4, linc-CHMP2B-1, linc-CXADR-2, linc-LOC285033-5, linc-ARHGAP28-2, linc-RGMA-7, linc-BMS1-3, linc-Clorf86, linc-CASP10-1, linc-SYNPO2-2, linc-FAM71F2-1, linc-TNKS-3, linc-MMADHC-3, linc-LEPROTL1-6, linc-BCL2L10, linc-OR5AU1, linc-SH3BGRL2-1, linc-TRIM43B-2, linc-ALG2-5, linc-C8orf79-2, linc-CEBPB-1, linc-RPS14, linc-RRP12, linc-FAM98A-3, linc-TACC3, linc-MPPE1-4, linc-IDH3B-1, linc-C8orf79-4, linc-DEFB105B-3, linc-HSF2-1, linc-EPT1, linc-RPGRIP1-3, linc-MUC20-1, linc-MAGI2-3, linc-SNTG2-3, linc-DEFB105B-2, linc-TCP10-4, linc-NAV3-1, linc-CSTB-6, linc-TCF7L2-3, linc-SPNS3, linc-FURIN, linc-HNRNPA1-3, linc-C1orf63, linc-SPAST-2, linc-EGFL7-1, linc-THBS2-3, linc-FRMPD1, linc-ITGB2-3, linc-TRIP11, linc-FER-1, linc-TMEM132D-1, linc-C14orf4-2, linc-GPATCH2-9, linc-ZNF236-4, linc-TTLL7-2, linc-APBA2-3, linc-ZNF32-5, linc-ALDH1A3-1, linc-GBP5-2, linc-DYNC1I1-2, linc-NR2F2-3, linc-B3GAT2-4, linc-CEP57-3, linc-CPPED1-3, linc-PHYHIPL, linc-C9orf170-1, linc-SPACA3, linc-FAM103A1, linc-INHBB, linc-ANKRD20A1-2, linc-CRP, linc-CPEB2-16, linc-PLCH2, linc-SHISA6-1, linc-ODZ3-5, linc-Clorf43-2, linc-HIST1H2AI-1, linc-BEND7-1, linc-CTSD-3, linc-RBKS-1, linc-PRSS38, linc-HAAO-6, linc-SLC5A9-4, linc-ZEB2-7, linc-FAM75A6-7, linc-DCT-2, linc-LY75, linc-DKK3-3, linc-TRPM5, linc-USPL1-1, linc-TPT1-2, linc-OBP2B, linc-C5orf38-4, linc-MMEL1-3, linc-GALNTL5-3, linc-ZDHHC6-2, linc-C5orf38-5, linc-HAAO-4, linc-CXorf36-3, linc-WDTC1, linc-LOC100129335-2, linc-DYNC2H1-4, linc-PEPD-1, linc-HDDC2-4, linc-TPT1-1, linc-USP16-5, linc-PICK1, linc-RHOXF1-3, linc-OXCT1-1, linc-BOD1-2, linc-ARHGEF37-2, linc-AQPEP, linc-PABPC4L-1, linc-MAP1LC3B-5, linc-TOX3-2, linc-FRMD6-2, linc-DEK-6, linc-ZFP64-5, linc-PRKAA2-8, linc-ABCA5-7, or linc-PRKACG-2.

In yet another example, the DNMT1-associated RNA that is under expressed in the cancer cells can include at least one of linc-AP2B1-2, linc-UTRN, linc-GPR65-6, linc-EGFL7-1, linc-ZNF404, linc-FRG1-5, linc-BCL2L10, linc-GIMAP8-1, linc-MAGI2-3, linc-DEFB105B-2, linc-C1orf43-2, linc-RGMA-7, linc-SHISA6-1, linc-SYT4-1, linc-GRPEL1-1, linc-ZNF583-1, linc-ZEB2-7, linc-GPATCH2-9, linc-ARHGEF37-2, linc-GTPBP8, and combinations thereof.

In some embodiments, a nucleic acid encoding the DNMT1-associated RNA can be substantially homologous or have a sequence identity that is substantially identical to native (or nonmutated) DNMT1-associated RNA such that when the nucleic acid encoding the DNMT1-associated RNA is administered to cancer cells of the subject, cancer growth, proliferation and/or metastasis is inhibited or reduced. By substantially homologous, it is meant the DNMT1-associated RNA has an at least about 80%, about 90%, about 95%, about 96%, about 97%, about 98%, about 99% or about 100% sequence identity with the nucleotide sequence of the native (or nonmutated) DNMT1-associated RNA.

In some embodiments, a nucleic acid encoding the downregulated DNMT1-associated RNA can have a nucleic acid sequence substantially homologous to the DNMT1-associated RNA or corresponding nucleic acid sequence listed in Tables 1, 3, and 5. For example, the nucleic acid can have a nucleic acid sequence substantially homologous to the nucleic acid sequence of linc-SMAD3. The nucleic encoding the DNMT1-associated RNA can be administered to cells through gene therapy using, for example, a nucleic acid construct. In general, there are two approaches to gene therapy in humans. For in vivo gene therapy, a nucleic acid construct encoding the nucleic acid or polynucleotide of interest can be administered directly to the subject or cells. Alternatively, in ex vivo gene therapy, cells are removed from the subject and treated with a nucleic acid construct to express the gene of interest. In the ex vivo method of gene therapy, the treated cells are then re-administered to the patient.

Numerous different methods for gene therapy are well known in the art. These methods include, but are not limited to, the use of nucleic acid constructs provided in DNA plasmid vectors as well as DNA and RNA viral vectors. These vectors are engineered to express DNMT1-associated RNA when integrated into patient cells.

Additionally, nucleic acid constructs for use in methods described herein may have expression signals, such as a strong promoter, a strong termination codon, adjustment of the distance between the promoter and the cloned gene, and the insertion of a transcription termination sequence.

In certain aspects, the nucleic acid construct includes a nucleic acid substantially homologous to DNMT1-associated RNA operably linked to a promoter to facilitate DNMT1-associated RNA expression within a cancer cell. The promoter may be a strong, viral promoter that functions in eukaryotic cells such as a promoter derived from cytomegalovirus (CMV), simian virus 40 (SV40), mouse mammary tumor virus (MMTV), Rous sarcoma virus (RSV), or adenovirus.

Alternatively, the promoter used may be tissue-specific, cell type-specific promoter, or a strong general eukaryotic promoter, such as the actin gene promoter. In another aspect, the promoter is a regulated promoter, such as a tetracycline-regulated promoter, expression from which can be regulated by exposure to an exogenous substance (e.g., tetracycline).

Introduction of one or more of the nucleic acid construct(s) including a nucleic acid encoding a DNMT1-associated RNA can be achieved using a variety of gene transfer protocols permitting transfection of the nucleic acid construct into the cells. Genetic change can be accomplished either by incorporation of the new nucleic acid into the genome of the host cell, or by transient or stable maintenance of the new DNA as an episomal element. A cell has been “transfected” when the nucleic acid construct has been introduced inside the cell membrane using any technology used to introduce nucleic acid molecules into cells.

A number of transfection techniques are well known in the art and are disclosed herein. See, for example, Graham et al., Virology, 52: 456 (1973); Sambrook et al., Molecular Cloning, a laboratory Manual, Cold Spring Harbor Laboratories (New York, 1989); Davis et al., Basic Methods in Molecular Biology, Elsevier, 1986; and Chu et al., Gene, 13: 197 (1981). Such techniques can be used to introduce one or more nucleic acid constructs described herein into the cells.

In some aspects, the nucleic acid construct can be introduced into cancer cells using a viral vector. The precise vector and vector formulation used will depend upon several factors, such as the size of the nucleic acid construct to be transferred and the delivery protocol to be used. The nucleic acid construct can also be introduced as infectious particles, e.g., DNA-ligand conjugates, calcium phosphate precipitates, and liposomes.

In general, viral vectors used are composed of a viral particle derived from a naturally occurring virus, which has been genetically altered to render the virus replication-defective and to deliver a recombinant gene of interest for expression in a target cell. Numerous viral vectors are well known in the art, including, for example, retrovirus, lentivirus, adenovirus, adeno-associated virus, herpes simplex virus (HSV), cytomegalovirus (CMV), vaccinia and poliovirus vectors. The viral vector may be selected according to its preferential infection of the cells targeted.

Where a replication-deficient virus is used as the viral vector, the production of infectious virus particles containing either DNA or RNA corresponding to the nucleic acid construct can be achieved by introducing the viral construct into a recombinant cell line, which provides the missing components essential for viral replication. Transformation of the recombinant cell line with the recombinant viral vector will not result in production or substantial production of replication-competent viruses, e.g., by homologous recombination of the viral sequences of the recombinant cell line into the introduced viral vector. Methods for production of replication-deficient viral particles containing a nucleic acid of interest are well known in the art and are described in, for example, Rosenfeld et al., Science 252:431-434, 1991 and Rosenfeld et al., Cell 68:143-155, 1992 (adenovirus); U.S. Pat. No. 5,139,941 (adeno-associated virus); U.S. Pat. No. 4,861,719 (retrovirus); and U.S. Pat. No. 5,356,806 (vaccinia virus).

In other embodiments, the nucleic acid construct including a nucleic acid encoding a DNMT1-associated RNA may be introduced into a cell using a non-viral vector. “Non-viral vector” as used herein is meant to include naked RNA (e.g., RNA not contained within a viral particle, and free of a carrier molecules such as lipids), chemical formulations comprising naked nucleic acid (e.g., a formulation of RNA (and/or DNA) and cationic compounds (e.g., dextran sulfate, cationic lipids)), and naked nucleic acid mixed with an adjuvant, such as a viral particle (e.g., the DNA or RNA of interest is not contained within the viral particle, but the formulation is composed of both naked DNA and viral particles (e.g., adenovirus particles) (see, e.g., Curiel et al. 1992 Am. J. Respir. Cell Mol. Biol. 6:247-52). Thus, “non-viral vector” can include vectors composed of nucleic acid plus viral particles where the viral particles do not contain the nucleic acid construct within the viral genome.

In some aspects, a liposome non-viral vector can be used to introduce the nucleic acid encoding the DNMT1-associated RNA into the cell. Liposomes for use in the method described herein can include a mixture of lipids, which bind to the nucleic acid construct and facilitate delivery of the construct into the cell. Examples of liposomes that can be used include DOPE (dioleyl phosphatidyl ethanol amine), CUDMEDA (N-(5-cholestrum-3-β-ol 3-urethanyl)-N1,N1-dimethylethylene diamine).

The nucleic acid encoding the DNMT1-associated RNA or vector thereof can be incorporated into pharmaceutical compositions suitable for administration to a subject. In some particular embodiments, the pharmaceutical composition comprises the vectors described herein and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances, such as wetting or emulsifying agents, preservatives or buffers, which enhance the shelf life or effectiveness of the vector or pharmaceutical composition.

The compositions described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form used depends on the intended mode of administration and therapeutic application. Typical compositions are in the form of injectable or infusible solutions.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the vector in the required amount in an appropriate solvent with one or a combination of ingredients enumerated above, as required, followed by filtered sterilization.

Generally, dispersions are prepared by incorporating the vector into a sterile vehicle that contains a basic dispersion medium and the required other ingredients from those enumerated above. In the case of sterile lyophilized powders for the preparation of sterile injectable solutions, the methods of preparation can include vacuum drying and spray-drying that yields a powder of the active ingredient plus any additional desired ingredient from a previously sterile-filtered solution thereof. The proper fluidity of a solution can be maintained, for example, by the use of a coating, such as lecithin, by the maintenance of the required particle size in the case of dispersion and by the use of surfactants. Prolonged absorption of injectable compositions can be achieved by including an agent in the composition that delays absorption, for example, monostearate salts and gelatin.

The vectors described herein can be administered by a variety of methods known in the art. As will be appreciated by the skilled artisan, the route and/or mode of administration will vary depending upon the desired results. In certain embodiments, the vector may be prepared with a carrier that will protect the vector against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art.

In some embodiments, one or more agents that decrease the level of DNMT1-associated RNA that is upregulated in the cancer cells and/or agents that increase the level of DNMT1-associated RNA that is downregulated in the cancer cells can be administered to cancer cells of the subject at an amount effective to modulate the level of DNMT1-associated RNA and/or the interaction of DNMT1-associated RNA and DNMT1 in the cancer cells of the subject and treat cancer. The cancer can be, for example, breast cancer, such as metastatic breast cancer. In some embodiments, the breast cancer is primary breast cancer. In other embodiments, the cancer is prostate cancer, or colon cancer, or hepatocellular carcinoma.

It will be appreciated that the one or more agents that decrease the level of DNMT1-associated RNA that is upregulated in the cancer cells and/or agents that increase the level of DNMT1-associated RNA that is downregulated in the cancer cells can be used to treat other cancers, such as, small or non-small cell lung, oat cell, papillary, bronchiolar, squamous cell, transitional cell, Walker), leukemia (e.g., B-cell, T-cell, HTLV, acute or chronic lymphocytic, mast cell, myeloid), histiocytoma, histiocytosis, Hodgkin disease, non-Hodgkin lymphoma, plasmacytoma, reticuloendotheliosis, adenoma, adenocarcinoma, adeno-fibroma, adenolymphoma, ameloblastoma, angiokeratoma, angiolymphoid hyperplasia with eosinophilia, sclerosing angioma, angiomatosis, apudoma, branchioma, malignant carcinoid syndrome, carcinoid heart disease, carcinosarcoma, colon cancer, prostate cancer, cementoma, cholan-gioma, cholesteatoma, chondrosarcoma, chondroblastoma, chondrosarcoma, chordoma, choristoma, craniopharyngioma, chrondroma, cylindroma, cystadenocar-cinoma, cystadenoma, cystosarcoma phyllodes, dysgerminoma, ependymoma, Ewing sarcoma, fibroma, fibrosarcoma, giant cell tumor, ganglioneuroma, glioblastoma, glomangioma, granulosa cell tumor, gynandroblastoma, hamartoma, hemangioendo-thelioma, hemangioma, hemangiopericytoma, hemangiosarcoma, hepatoma, hepatocellular cancer, islet cell tumor, Kaposi sarcoma, leiomyoma, leiomyosarcoma, leukosarcoma, Leydig cell tumor, lipoma, liposarcoma, lymphangioma, lymphangiomyoma, lymphangiosarcoma, medulloblastoma, meningioma, mesenchymoma, mesonephroma, mesothelioma, myoblastoma, myoma, myosarcoma, myxoma, myxosarcoma, neurilemmoma, neuroma, neuro-blastoma, neuroepithelioma, neurofibroma, neurofibromatosis, odontoma, osteoma, osteosarcoma, papilloma, paraganglioma, paraganglioma nonchromaffin, pinealoma, rhabdomyoma, rhabdomyosarcoma, Sertoli cell tumor, teratoma, cell tumors, and other diseases in which cells have become dysplastic, immortalized, or transformed.

Other embodiments described herein relate to compositions and methods for measuring the levels of DNMT1-associated RNA described herein to analyze tissue of a subject having or suspected of having cancer, predict whether a subject has cancer or an increased risk of cancer, determine cancer prognosis in a subject, and/or monitor a subject's response to a treatment regimen for cancer. For example, a biological sample (e.g., a tumor sample) can be obtained from a subject and the level of at least one DNMT1-associated RNA selected from Tables 1, 2, 3, 4, 5, and 6 can be determined or measured from the sample of tissue to generate a DNMT1-associated RNA expression profile. The expression profile from the sample is then compared to an expression profile of a control or standard. A decrease in the expression of the at least one DNMT1-associated RNA selected from Table 1, 3, or 5 and/or increase in the expression of the at least one DNMT1-associated RNA selected from Table 2, 4, or 6 is indicative of the subject having cancer or an increased risk of cancer.

Measuring methods include any method of nucleic acid detection, for example in situ hybridization for DNMT1-associated RNA using antisense DNA or RNA oligonucleotide probes, ultra-high throughput sequencing, Nanostring technology, microarrays, rolling circle amplification, proximity-mediated ligation, PCR, qRT-PCR ChIP, ChIP-qPCR or antibodies, or protein or nucleic acid measurements. Comparatively high levels of DNMT1-associated RNA compared to control levels in normal cells can indicate metastasis or poor cancer prognosis. Similarly, comparatively low levels of DNMT1-associated RNA compared to control levels in normal cells may indicate cancer progression.

Information on levels of a given set of DNMT1-associated RNA obtained using biological samples from individuals afflicted with or at risk of cancer may be grouped to form an expression profile map. The expression profile map can result from the study of a large number of individuals with the same cancer or cancer sub-type. In certain embodiments, a cancer expression profile map is established using samples from individuals with matched age, sex, and body index. Each expression profile map provides a template for comparison to DNMT1-associated RNA expression patterns generated from unknown biological samples. DNMT1-associated RNA expression profile maps may be presented as a graphical representation (e.g., on paper or a computer screen), a physical representation (e.g., a gel or array) or a digital representation stored in a computer-readable medium.

As will be appreciated by those of ordinary skill in the art, sets of biomarkers whose expression profiles correlate with cancer may be used to identify, study, or characterize unknown biological samples. Accordingly, in one aspect, methods for characterizing or analyzing biological samples obtained from a subject suspected of having cancer, for diagnosing cancer in a subject, and for assessing the responsiveness of cancer in a subject to treatment are contemplated. In such methods the DNMT1-associated RNA expression levels determined for a biological sample, obtained from the subject, are compared to the levels in one or more control samples. The control samples may be obtained from a healthy individual (or a group of healthy individuals), and/or from an individual (or group of individuals) afflicted with cancer. As mentioned above, the control expression levels of the DNMT1-associated RNA of interest are preferably determined from a significant number of individuals, and an average or mean is obtained. In certain aspects, the levels determined for the biological sample under investigation are compared to at least one expression profile map for cancer, as described above.

The methods described herein may be applied to the study of any type of biological samples allowing one or more inventive DNMT1-associated RNA to be assayed. Examples of biological samples include, but are not limited to, blood, blood products (e.g., blood plasma), and tissue. In a particular aspect of the present invention, the biological sample is tissue or biopsy obtained from the subject.

The biological samples used in the practice of the inventive methods may be fresh or frozen samples collected from a subject, or archival samples with known diagnosis, treatment and/or outcome history. Biological samples may be collected by any non-invasive means. Preferably, there is enough of the biological sample to accurately and reliably determine the abundance of the set of DNMT1-associated RNA of interest. Multiple biological samples may be taken from the subject in order to obtain a representative sampling from the subject.

In some embodiments, the DNMT1-associated RNA are extracted from the biological sample before analysis. Methods of RNA extraction are well known in the art (see, for example, J. Sambrook et al., “Molecular Cloning: A Laboratory Manual”, 1989, 2nd Ed., Cold Spring Harbor Laboratory Press: Cold Spring Harbor, N.Y.). Most methods of RNA isolation from bodily fluids or tissues are based on the disruption of the tissue in the presence of protein denaturants to quickly and effectively inactivate RNAses. Isolated total RNA may then be further purified from the protein contaminants and concentrated by selective ethanol precipitations, phenol/chloroform extractions followed by isopropanol precipitation or cesium chloride, lithium chloride or cesium trifluoroacetate gradient centrifugations. Kits are also available to extract RNA (i.e., total RNA or mRNA) from bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Qiagen, Inc. (Valencia, Calif.).

In certain aspects, after extraction, lncRNA, lincRNA, or mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2nd Ed., Cold Spring Harbour Laboratory Press: New York; “Short Protocols in Molecular Biology”, F. M. Ausubel (Ed.), 2002, 5th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each probe being monitored, or using thermostable DNApolymerases (such as avian myeloblastosis virus reverse transcriptase or Moloney murine leukemia virus reverse transcriptase).

The diagnostic methods described herein generally involve the determination of the abundance levels of a plurality (i.e., one or more, e.g., at least 2, at least 3, at least 4, at least 5, at least 6, at least 7, at least 8, at least 9, at least 10 or more) of DNMT1-associated RNA in a biological sample obtained from a subject.

It will be appreciated that the diagnostic methods may involve determination of the expression levels of a set of DNMT1-associated RNA using any suitable method, including, but not limited to, polymerase chain reaction (PCR) (see, for example, U.S. Pat. Nos. 4,683,195; 4,683,202, and 6,040,166; “PCR Protocols: A Guide to Methods and Applications”, Innis et al. (Eds.), 1990, Academic Press: New York), reverse transcriptase PCR(RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320308), one-sided PCR (Ohara et al., Proc. Natl. Acad. Sci., 1989, 86: 5673-5677), in situ hybridization, Taqman based assays (Holland et al., Proc. Natl. Acad. Sci., 1991, 88:7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan™, and the like.

Nucleic acid probes for use in the detection of DNMT1-associated RNA in biological samples may be constructed using conventional methods known in the art. Suitable probes may be based on nucleic acid sequences encoding at least 5 sequential amino acids from regions of nucleic acids encoding a protein marker, and preferably comprise about 15 to about 50 nucleotides. A nucleic acid probe may be labeled with a detectable moiety, as mentioned above in the case of binding agents. The association between the nucleic acid probe and detectable moiety can be covalent or non-covalent. Detectable moieties can be attached directly to nucleic acid probes or indirectly through a linker (E. S. Mansfield et al., Mol. Cell. Probes, 1995, 9: 145-156). Methods for labeling nucleic acid molecules are well-known in the art (for a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35:135-153).

Nucleic acid probes may be used in hybridization techniques to detect DNMT1-associated RNA. The technique generally involves contacting an incubating nucleic acid molecules in a biological sample obtained from a subject with the nucleic acid probes under conditions such that specific hybridization takes place between the nucleic acid probes and the complementary sequences in the nucleic acid molecules. After incubation, the non-hybridized nucleic acids are removed, and the presence and amount of nucleic acids that have hybridized to the probes are detected and quantified.

Detection of DNMT1-associated RNA may involve amplification of specific polynucleotide sequences using an amplification method such as PCR, followed by analysis of the amplified molecules using techniques known in the art. Suitable primers can be routinely designed by one skilled in the art. In order to maximize hybridization under assay conditions, primers and probes employed in the methods of the invention generally have at least 60%, preferably at least 75% and more preferably at least 90% identity to a portion of nucleic acids encoding a protein marker.

Hybridization and amplification techniques described herein may be used to assay qualitative and quantitative aspects of expression of nucleic acid molecules comprising polynucleotide sequences coding for the inventive protein markers.

Alternatively, oligonucleotides or longer fragments derived from DNMT1-associated RNA may be used as targets in a microarray. A number of different array configurations and methods of their production are known to those skilled in the art (see, for example, U.S. Pat. Nos. 5,445,934; 5,532,128; 5,556,752; 5,242,974; 5,384, 261; 5,405,783; 5,412,087; 5,424,186; 5,429,807; 5,436,327; 5,472,672; 5,527,681; 5,529,756; 5,545,531; 5,554, 501; 5,561,071; 5,571,639; 5,593,839; 5,599,695; 5,624,711; 5,658,734; and 5,700,637). Microarray technology allows for the measurement of the steady-state level of large numbers of polynucleotide sequences simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; Chen et al., Genomics, 1998, 51: 313324; U.S. Pat. Nos. 5,143,854; 5,445,934; 5,807,522; 5,837, 832; 6,040,138; 6,045,996; 6,284,460; and 6,607,885).

Once the levels of the DNMT1-associated RNA of interest have been determined for the biological sample being analyzed, they are compared to the levels in one or more control samples or to at least one expression profile map for cancer described herein. Comparison of levels according to methods of the present invention is preferably performed after the levels obtained have been corrected for both differences in the amount of sample assayed and variability in the quality of the sample used. Correction may be carried out by normalizing the levels against reference genes (e.g., housekeeping genes) in the same sample. Alternatively or additionally, normalization can be based on the mean or median signal (e.g., Ct in the case of RT-PCR) of all assayed genes or a large subset thereof (global normalization approach).

For a given set of DNMT1-associated RNA, comparison of an expression pattern obtained for a biological sample against an expression profile map established for cancer may comprise comparison of the normalized levels on a biomarker-by-biomarker (DNMT1-associated RNA-by-DNMT1-associated RNA) basis and/or comparison of ratios of levels within the set of biomarkers.

Using methods described herein, skilled physicians may select and prescribe treatments adapted to each individual subject based on the diagnosis of a cancer provided to the subject through determination of the levels of the inventive DNMT1-associated RNA. In particular, the present invention provides physicians with a non-subjective means to diagnose cancer, which will allow for early treatment, when intervention is likely to have its greatest effect. Selection of an appropriate therapeutic regimen for a given patient may be made based solely on the diagnosis provided by the inventive methods. Alternatively, the physician may also consider other clinical or pathological parameters used in existing methods to diagnose cancer and assess its advancement.

In certain embodiments, the assays, methods and systems described herein relate to identifying a subject with cancer or a need for treatment for cancer. Certain embodiments are related to assays, methods and systems for identifying the severity of cancer in a sample, e.g., a biopsy sample, obtained from a subject. In some embodiments, where the level of DNMT1-associated RNA in the biological sample is at least about 2-fold, at least about 4-fold, at least about 8-fold, or at least about 10-fold increased (e.g., DNMT1-associated RNA of Table 2, 4, or 6) as compared to a reference DNMT1-associated RNA level, the subject is identified as likely to have cancer, and/or metastatic cancer. In other embodiments, where the level of DNMT1-associated RNA in the biological sample is at least about 2-fold, at least about 4-fold, at least about 8-fold, or at least about 10-fold decreased (e.g., DNMT1-associated RNA of Table 1, 3, or 5) as compared to a reference DNMT1-associated RNA level, the subject is identified as likely to have cancer, and/or metastatic cancer. In such instances, a subject identified as likely to have cancer, and/or metastatic cancer can be treated with a more aggressive anti-cancer treatment regimen.

In some embodiments, where the level of DNMT1-associated RNA in the biological sample is at least about 2-fold, at least about 4-fold, at least about 8-fold, or at least about 10-fold increased (e.g., DNMT1-associated RNA of Table 2, 4, or 6) as compared to a reference DNMT1-associated RNA level, the subject is predicted to have a poor outcome and low metastasis free survival, or a decreased survival chance as compared to a subject who has a DNMT1-associated RNA levels not statistically significant different or similar to reference DNMT1-associated RNA levels. In other embodiments, where the level of DNMT1-associated RNA in the biological sample is at least about 2-fold, at least about 4-fold, at least about 8-fold, or at least about 10-fold decreased (e.g., DNMT1-associated RNA of Table 1, 3, or 5) as compared to a reference DNMT1-associated RNA level, the subject is predicted to have a poor outcome and low metastasis free survival, or a decreased survival chance as compared to a subject who has a DNMT1-associated RNA levels not statistically significant different or similar to reference DNMT1-associated RNA levels. In such instances, a subject identified with a poor outcome and low metastasis free survival, or a decreased survival chance can be treated with a more aggressive anti-cancer treatment regimen.

In certain embodiments, the subject may be exhibiting a sign or symptom of cancer. In certain embodiments, the subject may be asymptomatic or not exhibit a sign or symptom of cancer, but can be at risk of developing cancer due to certain risk factors as described herein.

In some embodiments, the methods and assays described herein include (a) transforming the DNMT1-associated RNA into a detectable gene target; (b) measuring the amount of the detectable gene target; and (c) comparing the amount of the detectable gene target to an amount of a reference, wherein if the amount of the detectable gene target (e.g., DNMT1-associated RNA) is statistically different from that of the amount of the reference level for the gene target (e.g., DNMT1-associated RNA), the subject is identified as having cancer or is in need of a treatment for cancer.

In some embodiments, the reference can be a level of DNMT1-associated RNA in a normal healthy subject with no symptoms or signs of cancer or metastasis. For example, a normal healthy subject who does not have cancer. In some embodiments, the reference can also be a level of expression of DNMT1-associated RNA in a control sample, a pooled sample of control individuals or a numeric value or range of values based on the same. In some embodiments, the reference can also be a level of the biomarker in a tissue sample taken from non-cancerous tissue of the subject. In certain embodiments, wherein the progression of cancer in a subject is to be monitored over time, the reference can also be a level of DNMT1-associated RNA in a tissue sample taken from the tissue of the subject at an earlier date.

In certain embodiments, a DNMT1-associated RNA, such as listed in Tables 2, 4, and 6, is upregulated in a biological sample, e.g., a biopsy sample from a subject with cancer. If the level of DNMT1-associated RNA is higher than a reference level of that biomarker, the subject is more likely to have cancer or to be in need of a treatment for cancer. The level of a DNMT1-associated RNA, which is higher than a reference level for that DNMT1-associated RNA, by at least about 10% than the reference amount, at least about 20%, at least about 30%, at least about 40%, at least about 50%, at least about 80%, at least about 100%, at least about 200%, at least about 300%, at least about 500% or at least about 1000% or more, is indicative that the subject has cancer.

In other embodiments, a DNMT1-associated RNA, such as listed in Tables 1, 3, and 5, is downregulated in a biological sample, e.g., a biopsy sample from a subject with cancer. If the level of DNMT1-associated RNA is lower than a reference level of that biomarker, the subject is more likely to have cancer or to be in need of a treatment for cancer. The level of a DNMT1-associated RNA which is lower than a reference level for that DNMT1-associated RNA by at least about 10% than the reference amount, at least about 20%, at least about 30%, at least about 40%, at least about 50%, at least about 80%, at least about 100%, at least about 200%, at least about 300%, at least about 500% or at least about 1000% or more, is indicative that the subject has cancer.

In another embodiment, the assays can include a system for transforming and measuring the amount levels of DNMT1-associated RNA as described herein and comparing them to reference expression levels. If the comparison system, which can be a computer implemented system, indicates that the amount of the measured expression product is statistically different from that of the reference amount, the subject from which the sample is collected can be identified as having an increased risk for having cancer or for a subject in need of a treatment for cancer or metastasis.

Systems (and computer readable media for causing computer systems) for performing the methods can include (a) at least one memory containing at least one computer program adapted to control the operation of the computer system to implement a method that includes (i) a determination module configured to identify and detect at the level of DNMT1-associated RNA in a biological sample obtained from a subject; (ii) a storage module configured to store output data from the determination module; (iii) a computing module adapted to identify from the output data whether the level of DNMT1-associated RNA measured in the biological sample obtained from a subject varies by a statistically significant amount from the DNMT1-associated RNA level found in a reference sample and (iv) a display module for displaying whether the level of DNMT1-associated RNA or other markers measured has a statistically significant variation in level in the biological sample obtained from a subject as compared to the reference DNMT1-associated RNA level and/or displaying the relative expression levels of the biomarkers, e.g., DNMT1-associated RNA levels and (b) at least one processor for executing the computer program.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a determination module, a storage module, a computing module, and a display module. The functional modules can be executed on one, or multiple, computers, or by using one, or multiple, computer networks. The determination module has computer executable instructions to provide e.g., levels of expression products etc in computer readable form.

The determination module can comprise any system for detecting a signal elicited from the DNMT1-associated RNA described herein in a biological sample. In some embodiments, such systems can include an instrument, e.g., StepOnePlus Real-Time PCR systems (Applied Biosystems) as described herein for quantitative RT-PCR. In another embodiment, the determination module can comprise multiple units for different functions, such as amplification and hybridization. In one embodiment, the determination module can be configured to perform the quantitative RT-PCR methods described in the Examples, including amplification, detection, and analysis.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, alleleic variants, and frequency of each alleleic variant. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

The “computing module” can use a variety of available software programs and formats for computing the relative expression level of the DNMT1-associated RNA described herein. Such algorithms are well established in the art. A skilled artisan is readily able to determine the appropriate algorithms based on the size and quality of the sample and type of data. By way of an example, when the level of DNMT1-associated RNA in a biological sample obtained from a subject is measured, a comparison module can compare or match the output data—with a reference DNMT1-associated RNA level in a reference sample. In certain embodiments, the reference expression level can have been pre-stored in the storage module. During the comparison or matching process, the comparison module can determine whether the expression level in the tissue sample obtained from a subject is lower than the reference expression level to a statistically significant degree. In various embodiments, the comparison module can be configured using existing commercially-available or freely-available software for comparison purpose, and may be optimized for particular data comparisons that are conducted.

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In certain embodiments, the content displayed on the display module can indicate whether the DNMT1-associated RNA measured have a statistically significant variation in expression (e.g., increase or decrease) between the biological sample obtained from a subject as compared to a reference expression level. In certain embodiments, the content displayed on the display module can indicate the degree to which the DNMT1-associated RNA were found to have a statistically significant variation in expression between the biological sample obtained from a subject as compared to a reference expression level. In certain embodiments, the content displayed on the display module can indicate whether the subject has an increased risk of having cancer, and/or the severity of the cancer. In certain embodiments, the content displayed on the display module can indicate whether the subject is in need of a treatment for cancer. In certain embodiments, the content displayed on the display module can indicate whether the subject has an increased risk of having a more severe case of cancer or metastasis. In some embodiments, the content displayed on the display module can be a numerical value indicating one of these risk or probabilities. In such embodiments, the probability can be expressed in percentages or a fraction.

### Example

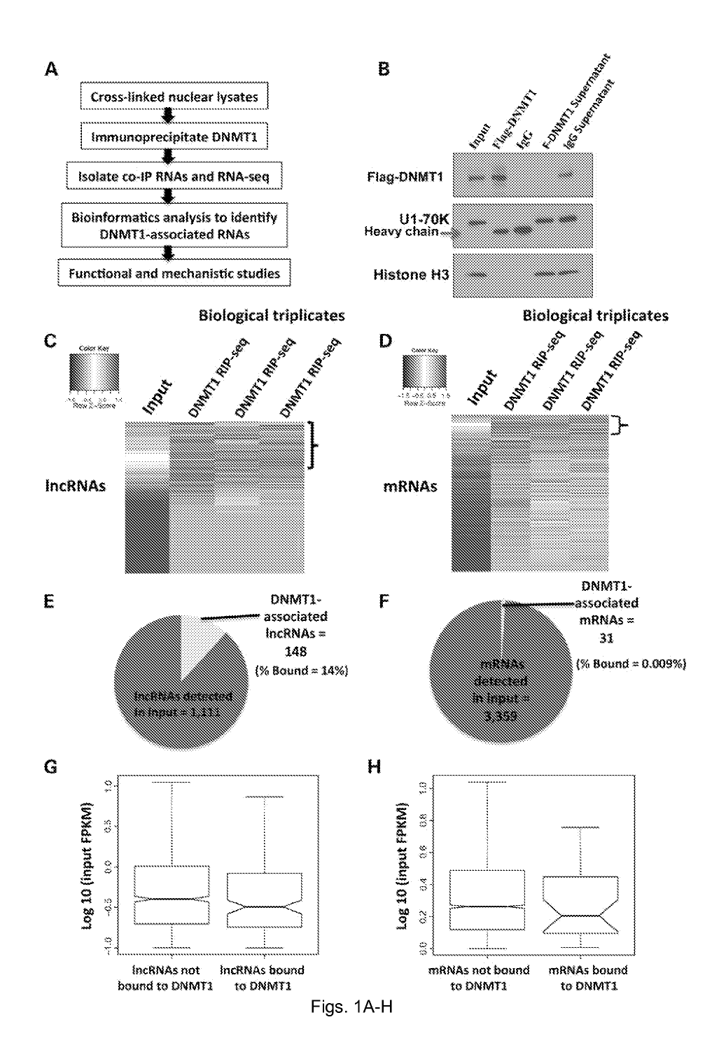

In this Example we identified specific interactions between a subset of human lncRNAs and the DNA methyltransferase DNMT1 using RNA co-immunoprecipitation (RIP) followed by next generation RNA sequencing (RIP-seq) (FIG. 1A). Analysis of one such lncRNA, TCONS_00023265, which we named DACOR1, revealed a critical role of this lncRNA in regulating DNA methylation and gene expression in colon cells. Furthermore, induction of DACOR1 is sufficient to suppress the growth of colon cancer cells by regulating the expression of specific genes and pathways including cellular metabolism. Our results suggest a potential new mechanism by which the human methylome is regulated in human health and disease.

### Material and Methods

### Optimization of RIP in HCT116 Cells

We have previously utilized RIP in human fibroblasts and HeLa cells to identify interactions between human lncRNAs and several chromatin-modifying complexes. For this study, we optimized our RIP protocol in HCT116 cells by initially performing control experiments on a well-conserved RNA-protein interaction in the spliceosome: the interaction between U1-70K protein and the small nuclear RNA U1. First, we tested an antibody against U1-70K in immunoprecipitation experiments and confirmed that this antibody specifically immunoprecipitates U1-70K protein from HCT116 cell lysate. We also used an IgG antibody that should not recognize any protein as a negative control. Subsequently, we performed three independent biological replicates of U1-70K RIPs from crosslinked HCT116 cell lysate. After several stringent washes, we reversed the formaldehyde crosslinking by heat and isolated associated RNA using Trizol. Quantitative Real-time PCR (qRTPCR) analysis of U1 RNA using three distinct endogenous controls (GAPDH, 18S rRNA and CLDN3) revealed a specific interaction between U1-70K and U1RNA. These results suggest that our RIP protocol is optimized in HCT116 to detect specific RNA-protein interactions.

### Immunoprecipitation (IP) of U1-70K and Flag-DNMT1 and Western Blot Analysis

We utilized an antibody against U1-70K (Synaptic Systems, Cat #203 001) to immunoprecipitate (IP) the U1-70K protein, and antiflag antibody to IP flag-DNMT1 from HCT116 cell lysates as follows: HCT116 cells were grown in 2×15 cm plates before harvesting by trypsin. An equal amount of media was added to quench the reaction, and the cells were collected by centrifugation in a 15-ml conical tube at 500 g for 10 min. The pellets were washed twice with PBS prior to fixing in a final concentration of 0.3% formaldehyde for 15 min at room temperature. The reaction was quenched by adding glycine to a final concentration of 0.125 mM and incubated at room temperature for 5 min. The cells were pelleted by spinning at 500 g for 10 min and then washed twice with 1×PBS before suspending the pellets in 2.2 ml of RIPA buffer (150 mM NaCl, 1% NP-40, 0.5% sodium deoxycholate, 0.1% SDS, 50 mM Tris-HCl (pH 7.4), 1 mM EDTA). The cells were incubated at 37° C. for 30 min and vortexed every 5 min at 30-s intervals for the duration of the incubation. Samples were homogenized using a dounce homogenizer to disrupt cellular membranes. The lysate was centrifuged using a microcentrifuge at maximum speed (˜13 300 RPM), and the supernatant was transferred to a new tube. A total of 100 μl of the supernatant was taken as input, and half of remaining supernatant was incubated with an antibody against protein of interest (i.e., U1-70K or flag-DNMT1), and the second half with an IgG antibody (negative control) overnight with rotation at 4° C. Next day, 50 μl of protein A/G magnetic beads was added to each tube and incubated for 30 min at room temperature with rotation. The beads, which now have the antibody and bound protein, were collected using a magnet and washed three times with RIPA buffer and once with 1×PBS. For protein analysis bywestern blot, we added 100 μl of Laemmli buffer to each tube and incubated the samples at 95° C. for 5 min before running the samples on a denaturing SDS-PAGE gel.

### RNA Co-Immunoprecipitation of U1-70K and Flag-DNMT1

The same protocol described earlier was utilized for RIP of U1-70K or flag-DNMT1 from HCT116 cells. However, for the isolation of co-immunoprecipitated RNAs, we suspended the magnetic beads+antibody+protein in 100 μl of buffer C (150 mM NaCl, 50 mM Tris-HCl (pH=7.4), 5 mM EDTA, 10 mM DTT, 1% SDS) and 10 μg of proteinase K. The samples were incubated at 42° C. for 30 min for protein digestion, and subsequently at 65° C. for 4 h to reverse the formaldehyde crosslinking. RNA was isolated by adding 800 μl of Trizol and 200 μl of chloroform to each sample, mixed and centrifuged at full speed for 10 min, and the upper clear layer (˜600 μl) was transferred to a 1.5-ml tube with 600 μl of 70% ethanol. The mixture was applied to an RNeasy mini kit column (Qiagen) according to the manufacturer's protocol. All samples were treated with DNase prior to final washes and elution with 20 μl of RNase-free water.

Analysis of RNA-Seq Data from RIP-Seq Samples

RNA-sequencing libraries were made using a stranded ScriptSeq V2 (Illumina) according to the manufacturer's protocol. Raw RNA-seq fastq files were aligned to UCSC human hg19 using TopHat v2.0.10. Transcript assembly was performed using Cufflinks v2.1.1. Relative transcript abundance for both mRNAs and lncRNAs was reported as fragments per kilobase of exon per million fragments mapped (fpkm). If fpkm values reported in the input sample were less than 1.0 for mRNAs and less than 0.1 for lncRNAs, the transcript was filtered as not expressed in HCT116 cells. Fold changes were then calculated as the average fpkm across RIP samples to the fpkm of the input control sample. Transcripts were identified as binding to DNMT1 if their fold change was greater than 2-fold. Heatmaps were generated using the heatmap function in the gplots package (version 2.12.1) in R [R Core Development Team. (2011) R: a language and environment for statistical computing. R Foundation for Statistical Computing, Vienna, Austria.]

### Quantitative Real-Time-PCR

RNA was converted to cDNA using RNA to cDNA EcoDry™ Premix Random Hexamers (Clontech). TaqMan assays for GAPDH, 18s rRNA, U1, CLDN3 and DACOR1 were purchased from Life Technologies. Other primer pairs were designed using primer3 software, and most primers used were designed to span exon-exon boundaries. TaqMan Mastermix (Life Technologies) or Maxima SyBr Green/ROX qPCR Master Mix (Thermo Scientific) was used for qRT-PCR. A comparative C_{T} quantitation was performed with a hold stage of 50° C. for 2 min and 95° C. for 10 min followed by 40× cycle of 95° C. for 15 s and 60° C. for 1 min and finally melt curve at 95° C. for 15 s, 60° C. for 1 min and a ramp to 95° C. at 0.3° C. increments. Analysis was done using the 2_ΔΔCT method with GAPDH as the reference gene.

### Colony Formation Assay

The colon cancer cell lines V481, V852, V866, V703 and V425 were transduced with either a control or DACOR1 lentivirus, and noninfected cells were eliminated by puromycin. For CFAs, cells were plated in either 6-well or 10-cm plates in triplicates of each condition (control versus DACOR1 lentivirus). Cells were plated at 1250, 2500, 5000 or 10 000 cells per well/plate and kept under puromycin selection. Colonies were fixed with methanol/acetic acid and subsequently stained with 0.1% crystal violet solution. Plates were scanned, and colonies were counted using the publically available ImageJ software. Average colony counts were calculated for control and DACOR1 plates for each cell line, and a paired t-test was used to test for statistical significance.

### Illumina 450K DNA Methylation Arrays

DNA was extracted from V481 and V852 cells using the DNeasy Blood and Tissue kit (Qiagen). DNA methylation profiling was performed at the Genomics Core Facility at Case Western Reserve University using the Illumina 450KHumanMethylation BeadChip (12 samples/chip). Biological triplicates from both the control and DACOR1 lentivirus-transduced cells were tested in order to detect accurate methylation status. Beta values, a ratio of the methylated/un-methylated signal, were reported and ranged from 0 (completely un-methylated) to 1 (completely methylated). In filtering probes, each cell line was analyzed separately. Reported beta values were removed if the P-value for detectable probe signal was >0.05. Targets were then filtered if only a single beta value remained in either condition. The median beta value was calculated for control and DACOR1 samples. Targets were further filtered if the difference in the maximum beta and minimum beta was >0.1 (10% different). Using the median beta, sites were determined as differentially methylated if the absolute value of the delta-beta was >0.1 (>10%).

### Next-Generation RNA Sequencing

Six RNA samples were isolated from V852 cells transduced with either a control lentivirus (n=3) or DACOR1 lentivirus (n=3). RNAs with RNA integrity number of >8 (max is 10) were considered high quality and suitable for RNA-seq. Library preparation was performed using Scriptseq™ Complete Gold (Human/Mouse/Rat) (Illumina) and sequenced on an Illumina Hi-Seq2500. All six samples were run on a single flow cell, and 100-bp paired-end strand specific sequencing reads were generated and mapped to human genome release hg19 using TopHat with two mismatches allowed for full-length reads. The raw reads were mapped to human genes annotated in Ref Seq database using Cufflinks V2.0.2, and CuffDiff was used for identifying differentially expressed genes. All expression values were calculated as fragment per kilo base of exon per million of mapped fragments (fpkm).

### ChIRP-Seq of DACOR1

The ChIRP-seq protocol was carried out as previously described by Chu et al. Briefly, 5×10⁸ V852 cells with DACOR1 lentivirus were first crosslinked using 1% formaldehyde for 10 min. The cells were spun down, suspended in Buffer A (Hepes 20 mM, KCl 10 mM, MgCl2 1.5 mM, DTT 0.5 mM, 1% Empigen) and dounced before collecting the nuclei by centrifugation. The nuclei were sonicated in nuclei lysis buffer (Tris-HCl pH 7.5, 20 mM, EDTA 10 mM, 1% SDS, 1 mM DTT, protease inhibitor cocktail, RNaseOut 80 U/ml) to produce 100- to 500-bp DNA fragments. LiCl₂ was added at 0.5 M to nuclear lysates. Equal amounts of nuclear lysates were incubated with either DACOR1-specific or non-specific DNA probes modified with a TEG linker and Biotin at their 5′ ends and incubated for 24 h at 37° C. with rotation. Next day, Ribo-Minus™ streptavidin-coated magnetic beads (Life Technologies) were blocked with 800 μg/ml yeast tRNA and 800 μg/ml BSA for 1 h at 37° C. in hybridization buffer (Tris-HCl pH 7.5 5 mM, EDTA 10 mM, LiCl2 500 mM) before washing and adding to nuclear lysates for 30 min. The beads were then washed three times with nuclear lysis buffer, wash buffer (Tris-HCl pH 7.5 5 mM, EDTA 0.5 mM, NaCl 1 M) and PBS. The beads were suspended in 200 μl of PBS and incubated at 75° C. for 5 min; the supernatant was collected from the beads and incubated at 65° C. overnight to reverse crosslinking before extracting DNA using DNeasy Blood & Tissue Kit (Qiagen). Paired-end DNA sequencing was performed on a HiSeq2000/2500 at Otogenetics Corporation. DNA reads were mapped against human genome (hg19) using Bowtie 2, and peak calling was performed by using MACS2. Peak annotation was completed using ChIPpeakAnno.

### PKM2 Activity Assay

Cells were collected by trypsinization, and pellets were washed twice by cold PBS. The pellets were then resuspended in RIPA buffer (150 mM NaCl, 1 mM EDTA, 1 mM DTT, 1% Triton X-100, 25.5 mM deoxycholic acid and 50 mM Tris-HCl, pH 7.5) and sonicated briefly at 4 C. The total extracts were subjected to PKM activity assay as follows: reaction mixtures contain 50 mM Tris-HCl pH 7.5, 100 mM KCl, 5 mM MgCl2, 0.5 mM ADP, 0.2 mM NADH, 8 units LDH (lactate dehydrogenase from sigma) and 1 mM DTT. The lysates (1-10 μg of total protein) were added to the assay mixture to reach 200 of the final volume in 96-well plates. The enzymatic reaction was initiated by the addition of PEP (phosphoenolpyruvic acid, 0.5 mM) as the substrate. The oxidation of NAPH was monitored at 340 nm for 3 mM using a Thermo Max microplate reader (Molecular Devices). The number of units of NADH oxidation was calculated using the standard extinction coefficient of NADH (ε=6.22 mM-1 cm⁻¹). This value was then divided by the total amount of protein added in the assay giving units per milligram of protein from the cell extracts. For all analyses, PKM2 activity was calculated using an amount of cell lysate where the reaction rates fell within the linear range of dependence on the concentration of lysate.

### Results

Identification of DNMT1-Associated lncRNAs in Colon Cancer Cells

We optimized our RIP protocol in the colon cancer cell line HCT116 and subsequently utilized it to identify potential interactions between DNMT1 and RNAs. As there are no reliable DNMT1 antibodies that are suitable for RIP applications, we utilized a knock-in DNMT1_3×-flag HCT116 cell line to overcome this limitation. First, we confirmed that DNMT1 is specifically immunoprecipitated, but not other abundant nuclear proteins such as U1-70K or histone H3 (FIG. 1B). To identify RNAs that potentially interact with DNMT1, we performed triplicate RIPs of DNMT1 using an anti-flag antibody and triplicate RIPs using an anti-IgG antibody as negative controls. We isolated co-immunoprecipitated RNAs and quantified the small amount of DNMT1-bound RNAs. We were able to generate RNA-seq libraries from DNMT1 RIPs but not from IgG RIPs, owing to depletion of non-specific RNAs by several stringent washes.

Three RNA-seq libraries from three independent biological replicates of DNMT1 RIPs were sequenced and mapped to the human genome (hg19). We also sequenced total nuclear RNA (input) from HCT116 cells as a control for our RIP experiments. We generated fpkm values for mRNAs and lncRNAs detected in the input sample and each of the three biological replicates of DNMT1 RIP-seq. The average fpkm of each transcript in the three biological replicates of DNMT1 RIP-seq was divided by the fpkm in the input sample to generate fold changes. We identified 148 lncRNAs (14% of lncRNAs detected in the input) and 31 mRNAs (0.009% of mRNAs detected in the input) as DNMT1-associated RNAs based on a 2-fold change or higher above input (FIG. 1C-F). We found the highest fold change of an lncRNA associated with DNMT1 to be ˜41-fold, whereas the highest fold change for an mRNA was only 7-fold, despite mRNAs being expressed at much higher levels than lncRNAs across all cell types. To rule out non-specific co-immunoprecipitation of highly abundant RNAs with DNMT1, we compared the expression of all DNMT1-bound versus DNMT1-unbound lncRNAs and mRNAs. We found that there was no expression bias of DNMT1-associated lncRNAs or mRNAs in comparison with unbound lncRNAs and mRNAs (FIG. 1G-H). Lastly, a close examination of DNMT1-associated mRNAs revealed that at least half of these mRNAs are poorly annotated transcripts with predicted open reading frames or miRNA precursors, suggesting that very few mRNAs associate with DNMT1. In summary, we have identified many lncRNAs and very small number of mRNAs that co-immunoprecipitate with DNMT1 in HCT116 cells by RIP-seq.

The DNMT1-Associated lncRNA, DACOR1, is Down-Regulated in Colon Cancer Cells

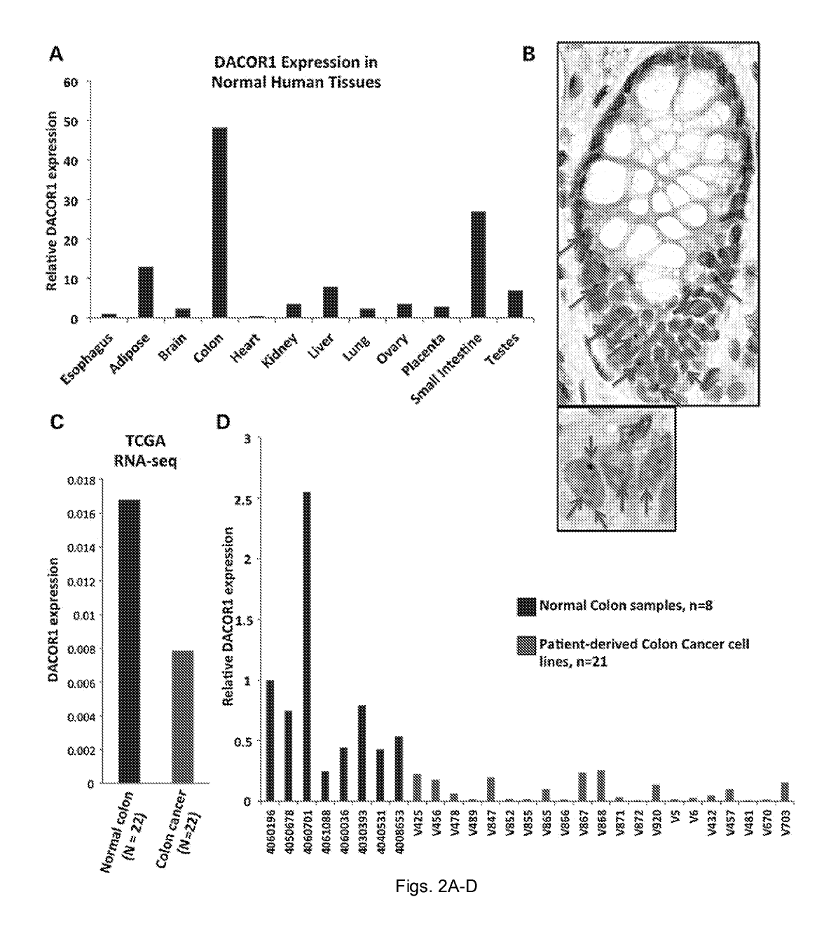

One DNMT1-associated lncRNA, designated TCONS_00023265, was of interest to us owing to its notable high, tissue-specific expression in normal colon tissues (FIGS. 2A and B) and repression in colon tumors and patient-derived colon cancer cell lines (FIGS. 2C and D). We therefore named this lncRNA DNMT1-associated Colon Cancer Repressed lncRNA 1 (DACOR1) (see below). In a panel of 12 human normal tissues, DACOR1 shows the highest expression in the colon as measured by qRT-PCR (FIG. 2A). We confirmed the expression of DACOR1 in the normal colon by RNA in situ hybridization and observed DACOR1 expression in the nuclei of colon crypts, the cells from which colon cancer originates (FIG. 2B, large panel). We also observed that DACOR1 occupies several discrete foci in the nucleus (FIG. 2B, small panel). Next, we examined DACOR1 expression in a cohort of 22 colon tumors in comparison with matched normal tissue based on RNA-seq data obtained from The Cancer Genome Atlas (TCGA). This analysis revealed that DACOR1 is down-regulated in colon tumors (FIG. 2C). We also examined the expression of the protein-coding gene SMAD3, the nearest coding gene to DACOR1, in the same TCGA cohort and found that SMAD3 shows variable expression in tumors versus normal colon. To further confirm that DACOR1 is down-regulated in colon cancer, we examined its expression by qRT-PCR in 8 normal colon samples and 21 patient-derived colon cancer cell lines with limited passage in culture (FIG. 2D). Several of the colon cancer cell lines displayed very low expression levels of DACOR1 that were barely detectable by qRT-PCR, further confirming the down-regulation of DACOR1 during colon tumorigenesis (FIG. 2D). These intriguing observations prompted us to further investigate the potential role of DACOR1 in colon cancer biology and its effects on DNA methylation and gene expression.

### DACOR1 Affects DNA Methylation Levels at Multiple Sites in the Human Genome

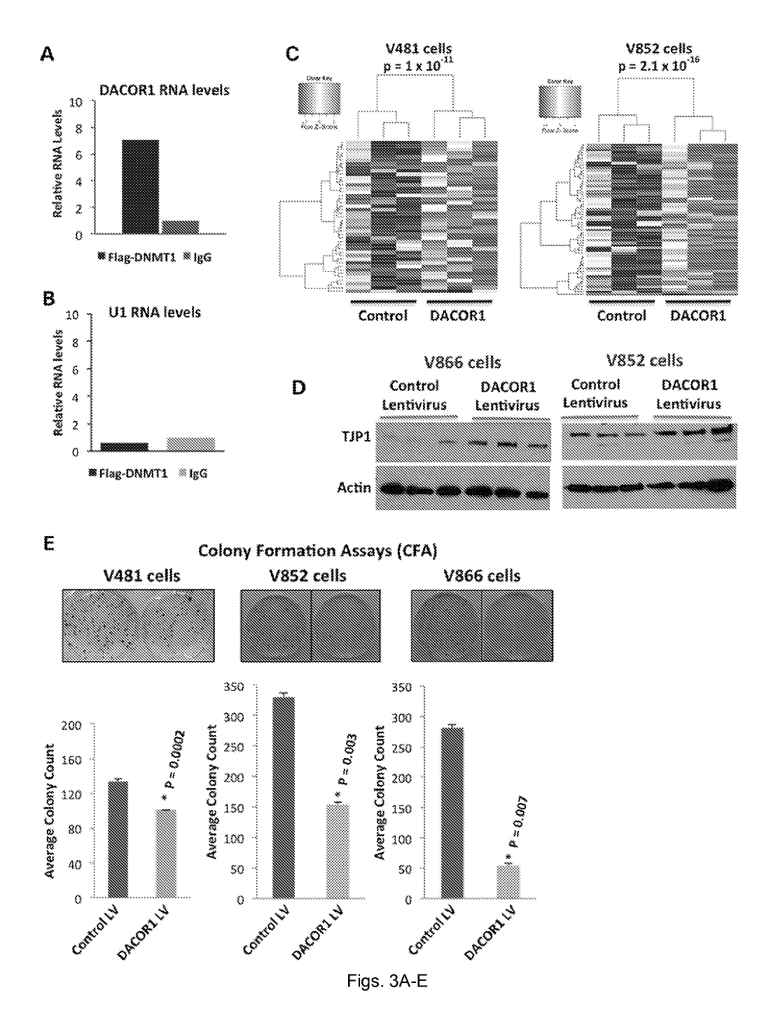

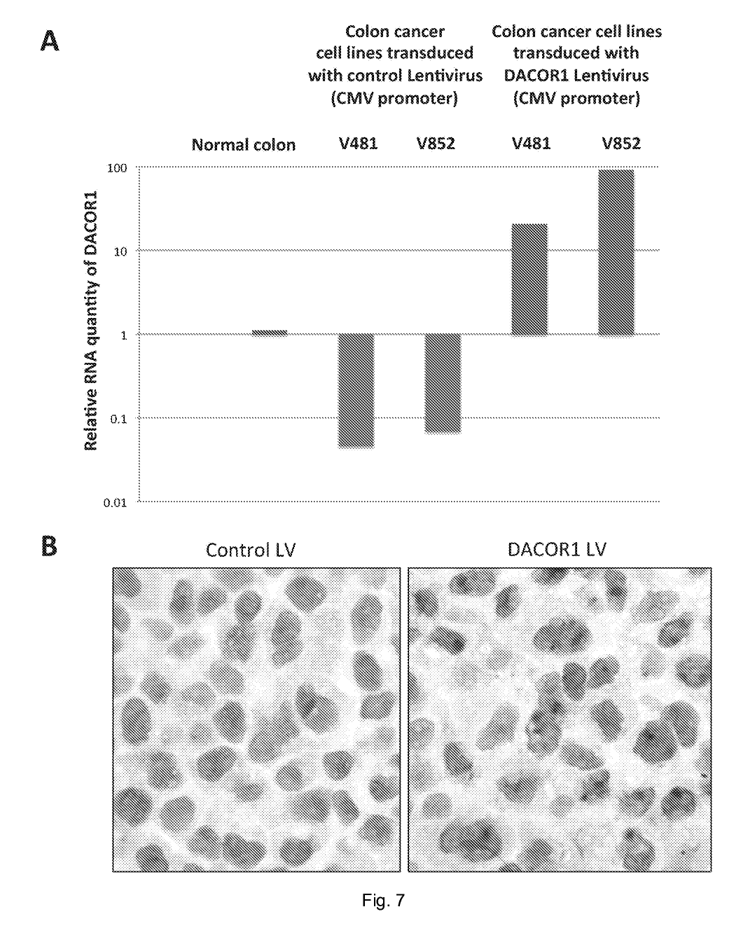

To determine the functional significance of DACOR1 association with DNMT1, we first validated the interaction of DACOR1 with DNMT1 in independent RIP experiments using RIP-qPCR (FIG. 3A). As a negative control, we examined the association of the highly abundant nuclear RNA U1 with DNMT1 and found no association (FIG. 3B). We then tested the effects of DACOR1 induction on DNA methylation in two distinct patient-derived colon cancer cell lines, V481 and V852. We transduced V481 and V852 cells with either a control or DACOR1 lentivirus, and the appropriate expression and nuclear localization of DACOR1 were confirmed by qRT-PCR and RNA in situ, respectively (FIGS. 7A and B). We isolated genomic DNA from these cell lines, and equal amount of DNA (1 μg) from each sample (n=12) was used for DNA methylation analysis using 450K DNA methylation arrays (Illumina). These arrays cover ˜500 000 CpG sites out of the 28 million CpG sites in the human genome. We identified 43 and 59 specific CpG sites in V481 and V852, respectively, which become differentially methylated in response to DACOR1 expression (FIG. 3C). Of these sites, 42/43 (in V481) and 58/59 (in V852) displayed a gain of DNA methylation (P<1×10-11 and 2.1×10-16, respectively). Next, we determined whether restoration of DACOR1 expression affected DNMT1 protein levels. We performed western blot analyses using a DNMT1 antibody in cells transduced with a control or DACOR1 lentivirus and found that DNMT1 protein levels were unchanged. In summary, DACOR1 induction appears to enhance DNA methylation at multiple loci without affecting DNMT1 protein levels.

### DACOR1 May Play a Role in Maintaining the Epithelial State of Colon Crypts

The high expression of DACOR1 in normal colon tissues and the localization of DACOR1 to colon crypts prompted us to examine its potential role in regulating the epithelial state of colon cells. To that end, we examined the effects of DACOR1 induction on the levels of key epithelial markers including Tight Junction Protein 1 (TJP1) and E-cadherin in two distinct colon cancer cell lines. We found that the expression of DACOR1 led to higher levels of TJP1 protein, but not E-Cadherin (FIG. 3D). To determine whether the change in TJP1 is at the transcriptional or post-transcriptional level, we measured TJP1 mRNA levels by qRT-PCR in three distinct colon cancer cell lines. We found DACOR1 expression to have no effect on TJP1 mRNA levels, suggesting that TJP1 protein levels are regulated posttranscriptionally by DACOR1 in colon cells. We also compared TJP1 mRNA levels in a cohort of 22 colon tumors versus 22 matched normal tissues from TCGA and found that TJP1 mRNA levels are not significantly affected in most patients, suggesting that TJP1 protein levels are regulated post-transcriptionally in colon tumors.

### DACOR1 Induction Reduces the Clonogenic Potential of Colon Cancer Cells

Our studies demonstrated that DACOR1 is down-regulated in colon tumors and patient-derived colon cancer cell lines, but the biological significance of this repression is yet to be determined. Normal colon crypts do not propagate in tissue culture, preventing us from performing knockdown experiments of DACOR1. We, therefore, examined the biological effects of DACOR1 by overexpressing it in several patient-derived colon cancer cell lines. Initially, we utilized three distinct patient-derived colon cancer cell lines (V481, V852 and V866) that we transduced with either a control or a DACOR1 lentivirus. Induction of DACOR1 in these patient derived colon cancer cell lines resulted in reduced growth of these cells. To quantify this effect, we performed colony formation assays (CFAs) using all three lines (V481, V852 and V866) and found that the induction of DACOR1 affected colony formation in V481 by ˜25% (P=0.0002), in V852 cells by ˜53% (P=0.003) and in V866 by 81% (P=0.007) (FIG. 3E). The effect of DACOR1 induction, although consistent in reducing colonies, varied among the three lines as each line was derived from a distinct patient tumor and thus has underlying genetic differences.

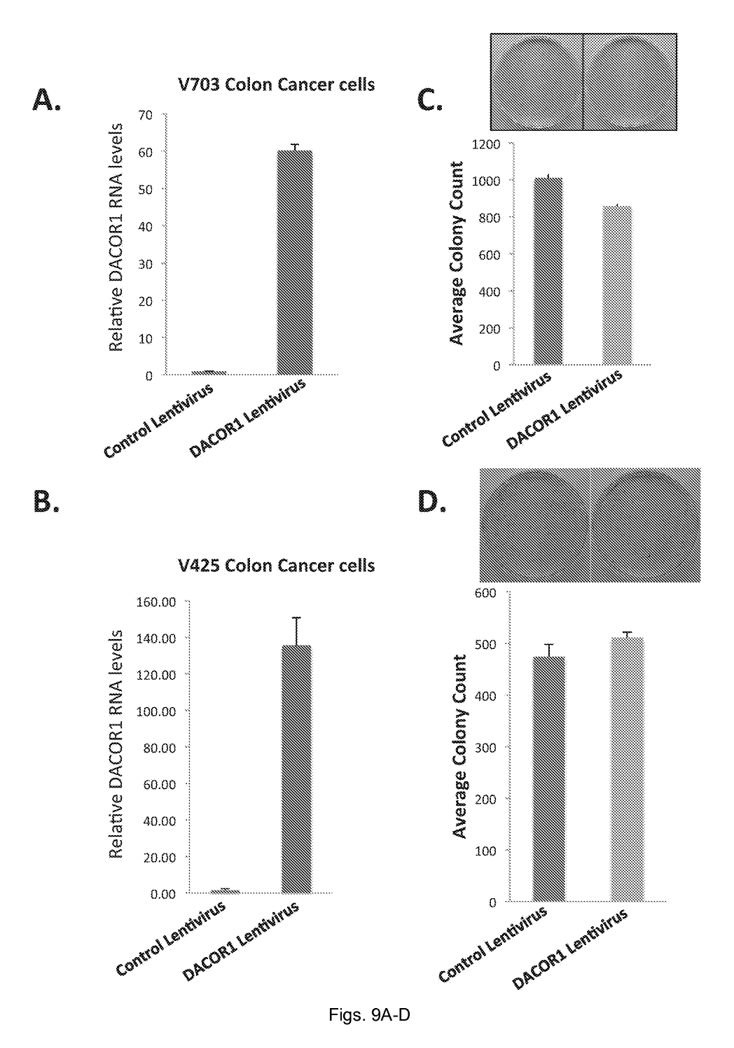

To test whether the effects we observed on colony formation were due to non-specific effects of overexpressing DACOR1, we performed several control experiments. First, we selected two patient-derived colon cancer cell lines, V703 and V425, that although had reduced levels of DACOR1 relative to normal colon; they still maintained some level of DACOR1 expression (FIG. 2D). Overexpression of DACOR1 in both cell lines had minor effects on colony formation of these cells, when compared with a control lentivirus (FIG. 9A-D). Second, to rule out that the phenotype is due to high expression levels of DACOR1 lentivirus (CMV promoter), we cloned the full length of DACOR1 downstream of a weak Pgk promoter and measured its expression levels in comparison with normal colon and control lentivirus. Using this approach, we are able to bring the overexpression level of DACOR1 closer to the expression levels observed in normal colon. We carried out CFAs of control versus DACOR1 lentivirus-transduced cells and also observed significant reduction in colony formation. Finally, we cloned the full length of an oncogenic lncRNA, TCON_00011938, which is not associated with DNMT1, downstream of a strong CMV promoter, and found that the overexpression of this distinct lncRNA led to increased colony formation. Collectively, these results suggest that DACOR1 induction reduces the clonogenic potential of colon cancer cells.

### DACOR1 Induction Affects Global Gene Expression of Colon Cancer Cells

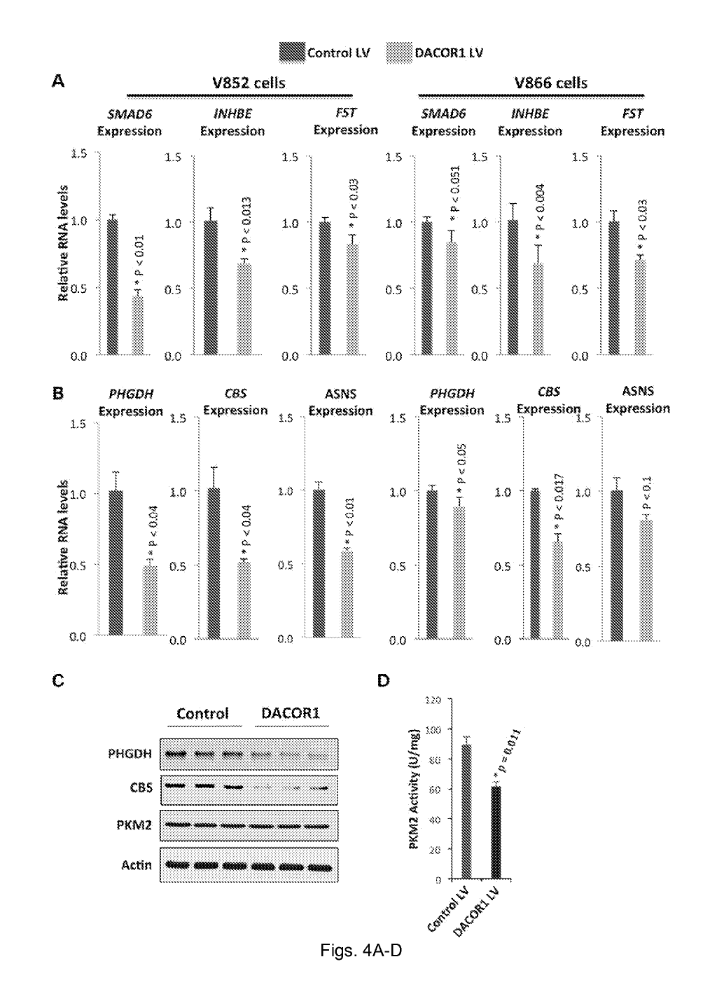

To gain insights into DACOR1 function, we performed RNA-seq using RNA isolated from the colon cancer cell line V852 transduced with either control or DACOR1 lentivirus and identified differentially expressed genes. We found that induction of DACOR1 affected the expression of 99 genes (P<0.05, q<0.05). Specifically, we observed that induction of DACOR1 led to the repression of several known inhibitors of TGF-β/BMP signaling, including SMAD6, INHBE (inhibin beta E) and FST (follistatin), which we confirmed by qRT-PCR in two distinct colon cancer cell lines (FIG. 4A). Previous studies have demonstrated that TGF-β/BMP signaling exerts a tumor-suppressor function in the colon, and it becomes inactivated or repressed in a majority of sporadic colorectal cancers. SMAD6, which is up-regulated in colon tumors and down-regulated by DACOR1, plays a major role in repressing TGF-β/BMP signaling.

We also found that the induction of DACOR1 led to the downregulation of several genes involved in amino acid metabolism with known roles in tumorigenesis, including PHGDH, PSAT1, CBS and ASNS. First, we confirmed that the induction of DACOR1 leads to the repression of these genes in two distinct colon cancer cell lines, V852 and V866, by qRT-PCR (FIG. 4B). We subsequently confirmed the repression of PHGDH at the protein level by western blot analysis (FIG. 4C). PHGDH plays a key role in de novo serine biosynthesis and is highly up-regulated in many colon tumors. To determine whether the repression of PHGDH by DACOR1 induction affects serine levels, we measured pyruvate kinase M2 (PKM2) activity, which is dependent on serine. Indeed, we found that DACOR1 induction leads to reduced PKM2 activity in two independent experiments (three replicates each) (FIG. 4D), without affecting overall PKM2 protein levels (FIG. 4C). Lastly, the repression of Cystathionine β-synthase (CBS) by DACOR1 is intriguing (FIG. 4C), as reduced CBS levels are known to lead to increased methionine, the substrate needed to generate S-adenosyl methionine (SAM). SAM is the key methyl donor utilized by DNA methyltransferases for DNA methylation in mammalian cells. Thus, DNMT1-DACOR1 interaction appears to indirectly regulate cellular SAM levels, and, consequently, genome-wide DNA methylation. Collectively, these findings suggest that DACOR1 plays key roles in regulating DNA methylation and specific tumor-suppressor and metabolic pathways in colon cells to potentially suppress colon tumorigenesis.

DACOR1 Interacts Directly with Chromatin at Specific Genomic Sites

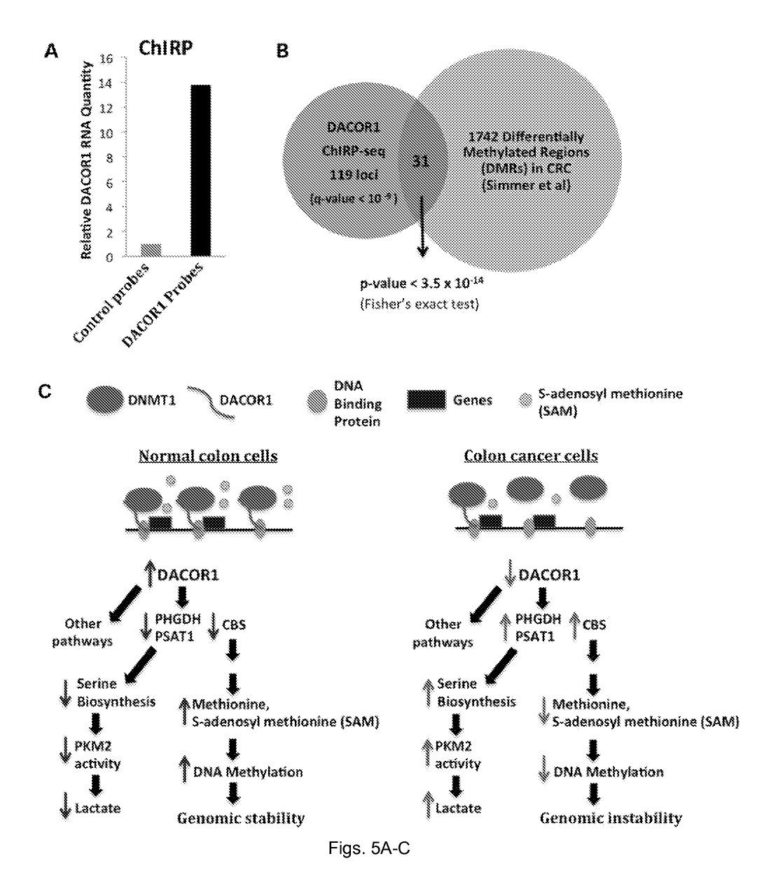

To gain insights into the potential mechanism(s) by which DACOR1 could regulate gene expression and consequently cellular phenotype, we mapped the genomic occupancy of DACOR1 across the entire human genome using ChIRP-seq. First, we designed several biotin-modified oligonucleotides complementary to DACOR1 and confirmed that we can specifically isolate DACOR1 from crosslinked cell lysates (FIG. 5A). Subsequent ChIRP-seq and analysis identified 338 DACOR1 genomic occupancy sites, including 161 peaks near 150 annotated genes (multiple peaks per gene in some cases) and 177 sites in intergenic regions. As expected, we observed a peak corresponding to the genomic region of DACOR1 transcription upstream of SMAD3, as we also captured newly synthesized DACOR1 transcripts. We compared the genomic occupancy sites of DACOR1 near annotated genes with differentially methylated regions (DMRs) in a cohort of colon tumors versus matched normal tissues. Of the 150 annotated gene loci occupied by DACOR1, 31 sites overlap with these DMRs (P<3.5×10-14) (FIG. 5B). These findings indicate that DACOR1 interacts with both DNMT1 and chromatin and, potentially, recruits and/or assembles the DNMT1 macromolecular protein complex at specific genomic sites to regulate epigenetic modifications and, consequently, the expression of specific genes and pathways (FIG. 5C).

The referenced patents, patent applications, and scientific literature, including accession numbers to GenBank database sequences, referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

1. A method for treating cancer in a subject in need thereof, the method comprising:
administering to cancer cells of the subject an agent effective to modulate the level of DNMT1-associated RNA and/or the interaction of DNMT1-associated RNA and DNMT1 in the cancer cells of the subject.

2. The method of claim 1, wherein the agent is effective to decrease the level of DNMT1-associated RNA that is over expressed in the cancer cells compared to normal cells.

3. The method of claim 1, wherein the cancer is selected from the group consisting of breast cancer and colon cancer.

4. The method of claim 1, wherein the DNMT1-associated RNA is DNMT1-associated long non-coding RNA.

5. The method of claim 1, wherein the DNMT1-associated RNA includes at least one of linc-GATA5-1, linc-FAM84B-9, linc-OR10H4, linc-DUSP26-6, linc-CCDC40-1, linc-CSPP1, linc-ASPSCR1, linc-U2AF1-5, linc-BEAN1, linc-EFR3A-7, linc-SLC25A45-5, linc-SEMA3A, linc-CXXC4-1, linc-EFR3A-4, linc-JAKMIP3-3, linc-KIAA1755-4, linc-EPHB4, linc-GAD1-1, linc-IGFBP2-3, linc-CCDC122-4, linc-NADSYN1-2, linc-DUSP26-1, linc-EFR3A-5, linc-TCF20, linc-RSPH1-1, linc-DUXA-2, linc-RTEL1, linc-INO80, linc-UBE3C-2, linc-STIM2-1, linc-VEZF1, linc-GPR183-2, linc-WHAMM-1, linc-FRMPD1, linc-MIB2, linc-SERTAD2-4, linc-HAAO-4, linc-CDH5-3, linc-NDUFAF2-3, linc-PPM1J, linc-LY6H, linc-MKLN1-2, linc-SERPIND1, linc-TCP10-5, linc-PPIAL4F-1, linc-BIRC7-3, linc-S100B-2, linc-C1QTNF9B, linc-PXN, linc-SRL, linc-ZNF692-6, linc-BDH1-3, linc-RALGAPB, linc-MYOD1, linc-OR4F16-9, linc-MUC20-3, linc-BTBD6-1, linc-CDK13-1, linc-ZNF8-2, linc-HIST1H2A1-2, linc-OR7C2-1, linc-MZF1-2, linc-CMPK1-3, linc-ARHGAP28-9, linc-NACC1, linc-BMS1-4, linc-TCP11L2-1, linc-CANX-1, linc-KCTD7-2, linc-TMEM105-2, linc-MRPS31, linc-RGL4-1, linc-METTL14-1, linc-NDUFB4-5, linc-ARF5-2, linc-NBPF15-1, linc-PHF10, linc-NADSYN1-1, linc-TMEM183B-1, linc-CALCOCO2-3, linc-BDH1-2, linc-ADAMTSL4, linc-RPS7-1, linc-ATP6V1C2-4, linc-FSCN2-1, linc-TUBGCP3-2, linc-HOXD1, linc-TGFBRAP1, linc-NOP14-3, linc-IER5L-2, linc-ASPRV1-1, linc-TPT1-2, linc-OAF-6, linc-COX5B-3, linc-ZBED1-4, linc-HIST1H2A1-1, linc-CALCOCO2-2, linc-ARF6-1, linc-MAP7-BP, linc-LOC285033-4, linc-ZNF674, linc-HTR5A-1, linc-GPR179, linc-RPP40, linc-SATB2-2, linc-MUC20-2, linc-ZNF516-4, linc-STX17, linc-CDH6-7, linc-SERHL2-3, or linc-OR4F16-4.

6. The method of claim 1, wherein the DNMT1-associated RNA includes at least one of linc-TMEM169-3, linc-HEATR6-2, linc-TM4SF4-2, linc-DACT2-3, linc-SAFB-2, linc-PTPRS-2, linc-ZPBP2, linc-MGAT4A, linc-DUSP26-5, linc-CA5A-2, linc-MERTK-2, linc-ABCA5-3, linc-GUCA2B, linc-HOXD1, linc-FOXA1-2, linc-EGLN1-2, linc-LRRC49-4, linc-TMEM18-13, linc-ANKRD27, linc-LAMA1-5, linc-TMEM183B-1, linc-UGDH, linc-PKMYT1, linc-PPP1R1B, linc-WFDC2-2, linc-DYNC1L11-2, linc-DHX37-22, linc-OAF-6, linc-ODF3B, linc-ARHGAP28-9, linc-DUSP26-6, linc-EVX2-8, linc-COPZ2, linc-DLGAP5-1, linc-XRCC4-3, linc-ZIC5, linc-KIN-5, linc-NACC1, linc-SERTAD2-4, linc-ASPSCR1, or linc-KIAA0232.

7. The method of claim 1, wherein the DNMT1-associated RNA includes at one of linc-DUSP26-6, linc-ASPSCR1, linc-SERTAD2-4, linc-ARHGAP28-9, linc-NACC1, linc-TMEM183B-1, linc-HOXD1, or linc-OAF-6.

8. The method of claim 2, wherein the agent comprises an RNA inhibitor of the DNMT1-associated RNA.

9. The method of claim 2, wherein the agent is selected from the group consisting of siRNA, miRNA, stRNA, snRNA, and antisense nucleic acid to the DNMT1-associated RNA.

10. The method of claim 1, wherein the agent is effective to increase the level of DNMT1-associated RNA that is under expressed in the cancer cells compared to normal cells.

11. The method of claim 1, wherein the cancer is selected from the group consisting of breast cancer and colon cancer.

12. The method of claim 1, wherein the DNMT1-associated RNA includes at least one of linc-SMAD3, linc-ANXA8L2-2, linc-TRAK1, linc-AP2B1-2, linc-UTRN, linc-TBX18, linc-GPR65-6, linc-STIL-2, linc-ENPP6-2, linc-GABRA5-6, linc-MRPS18C, linc-EGFL7-1, linc-KLHL31-2, linc-PSMA8-3, linc-ZNF404, linc-HMGB2, linc-OAF-4, linc-FRG1-5, linc-HIST1H3A, linc-TMEM56-3, linc-DBT-3, linc-GNAI1-2, linc-BCL2L10, linc-EPHA6-1, linc-PLDN, linc-GABRA5-5, linc-ACO1-2, linc-NEDD4L-1, linc-MTRNR2L1-2, linc-FAM155B, linc-GIMAP8-1, linc-MAGI2-3, linc-DHX37-17, linc-KLF6-3, linc-RAP1GAP2-1, linc-TMPRSS2-2, linc-C10orf57-3, linc-GPR157-3, linc-LAMA4-2, linc-STIM1, linc-RFC2-2, linc-MRGPRF-1, linc-DEFB105B-2, linc-CTDSP2-1, linc-PRPS1L1, linc-SLC19A1-4, linc-C1orf43-2, linc-COX4NB-8, linc-HES1-3, linc-FIGNL1, linc-OAF-2, linc-COX4NB-9, linc-FBXL5-2, linc-TMEM220-2, linc-KCNMB2-5, linc-KIAA0141, linc-DHX37-19, linc-RGMA-7, linc-ID2-1, linc-SHISA6-1, linc-SYT4-1, linc-TRIML2-5, linc-DHFRL1-4, linc-RGS9-1, linc-ODF2L, linc-SLC22A16, linc-ZPBP2, linc-AGMAT-3, linc-MT1B, linc-GRPEL1-1, linc-PFDN4-2, linc-OPRK1-4, linc-ZNF583-1, linc-PFDN4-3, linc-SAMSN1-3, linc-USP3-1, linc-SHISA6-2, linc-ADAM29-3, linc-ZEB2-7, linc-MLL5, linc-FOXF1-3, linc-BTBD3-3, linc-GPATCH2-9, linc-ARHGEF37-2, linc-KLF6-2, linc-CLMN-1, linc-FOXG1-4, linc-TAAR9-1, linc-GTPBP8, linc-ADAR, linc-SAFB-2, linc-CXorf49B-2, linc-SLCO2A1-1, linc-PTPRS-2, linc-EPCAM, linc-LPHN2-1, linc-AMN1, linc-FAM55D, linc-FAM75A6-4, or linc-PHOX2B-2.

13. The method of claim 1, wherein the DNMT1-associated RNA includes at least one of linc-GHRH, linc-VPS36-1, linc-C20orf79, linc-AUTS2-5, linc-ISLR2-3, linc-ZNF692-6, linc-IER31P1, linc-MANSC1, linc-CXADR-3, linc-ZFHX3-4, linc-ZNF404, linc-FAAH2-2, linc-CLRN2-1, linc-ATP6V1C2-3, linc-METTL14-2, linc-MAP1 LC3B-2, linc-TCP11L2-1, linc-GARS-1, linc-NOP14-3, linc-ANKRD55-6, linc-ARFIP1-8, linc-C17orf87, linc-AMAC1, linc-SYT4-1, linc-HTR1D, linc-WNT7B-2, linc-MAP1LC3B2-2, linc-TP53TG3B-6, linc-TMEM105-2, linc-MICB, linc-PLGLB2, linc-OR4F16-9, linc-RBM10, linc-KIAA1712-5, linc-CBLB-6, linc-ATG2B-2, linc-ADI1, linc-SPRY3-1, linc-BEAN1, linc-SHOX-5, linc-WIPF3, linc-SHOX-4, linc-DCAF17-1, linc-TNFRSF14, linc-GPR65-6, linc-PHF10, linc-ZNF692-5, linc-POLR3A-1, linc-LOC389493-2, linc-AP2B1-2, linc-LRRTM3-3, linc-SFMBT1, linc-BTBD6-1, linc-MTHFD2, linc-PRSS42, linc-RGMB-1, linc-ITIH2-10, linc-TPBG-3, linc-TMEM194A, linc-FRG2C-3, linc-ZKSCAN1-1, linc-HEATR2-2, linc-CDK13-1, linc-GIMAP8-1, linc-FAM101A-2, linc-IFITM5, linc-LRRTM3-2, linc-METTL14-1, linc-GTPBP8, linc-KLF13-1, linc-SLC5A3-2, linc-BET3L, linc-TUBGCP3-2, linc-CDH1, linc-PTGR2, linc-CDK17-4, linc-COG3-3, linc-CBR1-1, linc-CCR8-3, linc-LOC150786, linc-FRG1-5, linc-GABRA5-7, linc-DYDC1-5, linc-CREB1-1, linc-LEPROTL1-7, linc-C6orf145-3, linc-HIST1H2AG-4, linc-THSD4, linc-HS3ST3A1-1, linc-KLRC1, linc-ZNF583-1, linc-ZNF253-2, linc-UTRN, linc-ATP6V1C2-4, linc-GRPEL1-1, linc-TTC7A-2, linc-COG3-1, linc-IFLTD1, linc-GALNTL5-1, linc-PCM1, linc-ISLR2-2, linc-DHCR7-2, linc-HES5-2, linc-USP8, linc-VPS8-2, linc-RGL4-1, linc-CEBPG, linc-CRH-2, linc-DR1, linc-UBQLN2, linc-MTRNR2L9-3, linc-ID2-3, linc-TMED5, linc-RAB23-4, linc-NDFIP2-1, linc-ARHGAP5, linc-WRNIP1-2, linc-ANKRD20A1-14, linc-TLN2-1, linc-VPS8-3, linc-CNTNAP4, linc-CHMP2B-1, linc-CXADR-2, linc-LOC285033-5, linc-ARHGAP28-2, linc-RGMA-7, linc-BMS1-3, linc-C1orf86, linc-CASP10-1, linc-SYNPO2-2, linc-FAM71F2-1, linc-TNKS-3, linc-MMADHC-3, linc-LEPROTL1-6, linc-BCL2L10, linc-OR5AU1, linc-SH3BGRL2-1, linc-TRIM43B-2, linc-ALG2-5, linc-C8orf79-2, linc-CEBPB-1, linc-RPS14, linc-RRP12, linc-FAM98A-3, linc-TACC3, linc-MPPE1-4, linc-IDH3B-1, linc-C8orf79-4, linc-DEFB105B-3, linc-HSF2-1, linc-EPT1, linc-RPGRIP1-3, linc-MUC20-1, linc-MAGI2-3, linc-SNTG2-3, linc-DEFB105B-2, linc-TCP10-4, linc-NAV3-1, linc-CSTB-6, linc-TCF7L2-3, linc-SPNS3, linc-FURIN, linc-HNRNPA1-3, linc-C1orf63, linc-SPAST-2, linc-EGFL7-1, linc-THBS2-3, linc-FRMPD1, linc-ITGB2-3, linc-TRIP11, linc-FER-1, linc-TMEM132D-1, linc-C14orf4-2, linc-GPATCH2-9, linc-ZNF236-4, linc-TTLL7-2, linc-APBA2-3, linc-ZNF32-5, linc-ALDH1A3-1, linc-GBP5-2, linc-DYNC1I1-2, linc-NR2F2-3, linc-B3GAT2-4, linc-CEP57-3, linc-CPPED1-3, linc-PHYHIPL, linc-C9orf170-1, linc-SPACA3, linc-FAM103A1, linc-INHBB, linc-ANKRD20A1-2, linc-CRP, linc-CPEB2-16, linc-PLCH2, linc-SHISA6-1, linc-ODZ3-5, linc-C1orf43-2, linc-HIST1H2A1-1, linc-BEND7-1, linc-CTSD-3, linc-RBKS-1, linc-PRSS38, linc-HAAO-6, linc-SLC5A9-4, linc-ZEB2-7, linc-FAM75A6-7, linc-DCT-2, linc-LY75, linc-DKK3-3, linc-TRPM5, linc-USPL1-1, linc-TPT1-2, linc-OBP2B, linc-C5orf38-4, linc-MMEL1-3, linc-GALNTL5-3, linc-ZDHHC6-2, linc-C5orf38-5, linc-HAAO-4, linc-CXorf36-3, linc-WDTC1, linc-LOC100129335-2, linc-DYNC2H1-4, linc-PEPD-1, linc-HDDC2-4, linc-TPT1-1, linc-USP16-5, linc-PICK1, linc-RHOXF1-3, linc-OXCT1-1, linc-BOD1-2, linc-ARHGEF37-2, linc-AQPEP, linc-PABPC4L-1, linc-MAP1LC3B-5, linc-TOX3-2, linc-FRMD6-2, linc-DEK-6, linc-ZFP64-5, linc-PRKAA2-8, linc-ABCA5-7, or linc-PRKACG-2.

14. The method of claim 1, wherein the DNMT1-associated RNA includes at least one of linc-AP2B1-2, linc-UTRN, linc-GPR65-6, linc-EGFL7-1, linc-ZNF404, linc-FRG1-5, linc-BCL2L10, linc-GIMAP8-1, linc-MAGI2-3, linc-DEFB105B-2, linc-C1orf43-2, linc-RGMA-7, linc-SHISA6-1, linc-SYT4-1, linc-GRPEL1-1, linc-ZNF583-1, linc-ZEB2-7, linc-GPATCH2-9, linc-ARHGEF37-2, or linc-GTPBP8.

15. The method of claim 10, wherein the agent comprises DNMT1-associated RNA that is under expressed in the cancer cells compared to normal cells.

16. The method of claim 10, wherein the agent comprises an expression vector that encodes the DNMT1-associated RNA.

17-61. (canceled)
